SEQUENCE LISTING (52 sequences)

SEQ ID NO: 1 - protein, 5 aa, Artificial Sequence
  synthetic construct; Fab
SSRYR

SEQ ID NO: 2 - protein, 13 aa, Artificial Sequence
  synthetic construct; Fab
YGAYRLSSGVPYR

SEQ ID NO: 3 - protein, 6 aa, Artificial Sequence
  synthetic construct; Fab
GSSYPV

SEQ ID NO: 4 - protein, 7 aa, Artificial Sequence
  synthetic construct; Fab
NFSGSGI

SEQ ID NO: 5 - protein, 10 aa, Artificial Sequence
  synthetic construct; Fab
GSGSSRGYTR

SEQ ID NO: 6 - protein, 9 aa, Artificial Sequence
  synthetic construct; Fab
SGSGSRYAL

SEQ ID NO: 7 - protein, 5 aa, Artificial Sequence
  synthetic construct; Fab
YGYRS

SEQ ID NO: 8 - protein, 13 aa, Artificial Sequence
  synthetic construct; Fab
YSASGLYRGVPSR

SEQ ID NO: 9 - protein, 6 aa, Artificial Sequence
  synthetic construct; Fab
GYRSPV

SEQ ID NO: 10 - protein, 7 aa, Artificial Sequence
  synthetic construct; Fab
NLGSGYI

SEQ ID NO: 11 - protein, 10 aa, Artificial Sequence
  synthetic construct; Fab
SYRPSSGSTR

SEQ ID NO: 12 - protein, 10 aa, Artificial Sequence
  synthetic construct; Fab
SYSSRYSYAM

SEQ ID NO: 13 - protein, 5 aa, Artificial Sequence
  synthetic construct; Fab
SVSSA

SEQ ID NO: 14 - protein, 13 aa, Artificial Sequence
  synthetic construct; Fab
YSASSLYSGVPSR

SEQ ID NO: 15 - protein, 6 aa, Artificial Sequence
  synthetic construct; Fab
SYSSPI

SEQ ID NO: 16 - protein, 7 aa, Artificial Sequence
  synthetic construct; Fab
NLYSSSI

SEQ ID NO: 17 - protein, 10 aa, Artificial Sequence
  synthetic construct; Fab
SRSPRSGGTS

SEQ ID NO: 18 - protein, 10 aa, Artificial Sequence
  synthetic construct; Fab
RAAGMSTYGF

SEQ ID NO: 19 - protein, 5 aa, Artificial Sequence
  synthetic construct; Fab
YSYRS

SEQ ID NO: 20 - protein, 13 aa, Artificial Sequence
  synthetic construct; Fab
YRASRLYGGVPSR

SEQ ID NO: 21 - protein, 6 aa, Artificial Sequence
  synthetic construct; Fab
RSSYPV

SEQ ID NO: 22 - protein, 7 aa, Artificial Sequence
  synthetic construct; Fab
NFSGSSI

SEQ ID NO: 23 - protein, 10 aa, Artificial Sequence
  synthetic construct; Fab
YGSPGYGRTS

SEQ ID NO: 24 - protein, 11 aa, Artificial Sequence
  synthetic construct; Fab
SSYGSRSGYAM

SEQ ID NO: 25 - protein, 5 aa, Artificial Sequence
  synthetic construct; Fab
SGSSY

SEQ ID NO: 26 - protein, 13 aa, Artificial Sequence
  synthetic construct; Fab
YRASSLSSGVPSR

SEQ ID NO: 27 - protein, 6 aa, Artificial Sequence
  synthetic construct; Fab
YSSRLL

SEQ ID NO: 28 - protein, 7 aa, Artificial Sequence
  synthetic construct; Fab
NIYRYGI

SEQ ID NO: 29 - protein, 10 aa, Artificial Sequence
  synthetic construct; Fab
YGSPGSGRTY

SEQ ID NO: 30 - protein, 10 aa, Artificial Sequence
  synthetic construct; Fab
RYRYRYRSGL

SEQ ID NO: 31 - protein, 5 aa, Artificial Sequence
  synthetic construct; Fab
YSSRY

SEQ ID NO: 32 - protein, 13 aa, Artificial Sequence
  synthetic construct; Fab
YGAYGLYRGVPYR

SEQ ID NO: 33 - protein, 6 aa, Artificial Sequence
  synthetic construct; Fab
SYSYPI

SEQ ID NO: 34 - protein, 7 aa, Artificial Sequence
  synthetic construct; Fab
NLGYRYI

SEQ ID NO: 35 - protein, 10 aa, Artificial Sequence
  synthetic construct; Fab
SGSSGSGSTY

SEQ ID NO: 36 - protein, 10 aa, Artificial Sequence
  synthetic construct; Fab
GSSYSYRYAF

SEQ ID NO: 37 - DNA, 76 bp, Artificial Sequence
  synthetic construct; mutant Fab
agcaacaccaaggtcgacgccgccgttgcccccaaatcttgtgacaaactacacatagggccggccctctggttcc

SEQ ID NO: 38 - DNA, 45 bp, Artificial Sequence
  synthetic construct; mutant Fab
cttcatcttcccgccatctgatgcccagttgaaatctggaactgc

SEQ ID NO: 39 - DNA, 40 bp, Artificial Sequence
  synthetic construct; mutant Fab
agcagactacgagaaacacgccgtctacgcctgcgaagta

SEQ ID NO: 40 - protein, 6 aa, Artificial Sequence
  synthetic construct; Fab
SSYSSS

SEQ ID NO: 41 - protein, 7 aa, Artificial Sequence
  synthetic construct; Fab
SISSSSI

SEQ ID NO: 42 - protein, 10 aa, Artificial Sequence
  synthetic construct; Fab
YIYPSSGSTY

SEQ ID NO: 43 - protein, 9 aa, Artificial Sequence
  synthetic construct; Fab
YRYRSGHAL

SEQ ID NO: 44 - protein, 5 aa, Artificial Sequence
  synthetic construct; Fab
SSRYR

SEQ ID NO: 45 - protein, 13 aa, Artificial Sequence
  synthetic construct; Fab
YGAYRLSSGVPYR

SEQ ID NO: 46 - protein, 6 aa, Artificial Sequence
  synthetic construct; Fab
GSSYPV

SEQ ID NO: 47 - protein, 7 aa, Artificial Sequence
  synthetic construct; Fab
NPSGSGI

SEQ ID NO: 48 - protein, 10 aa, Artificial Sequence
  synthetic construct; Fab
GSGSSRGYTR

SEQ ID NO: 49 - protein, 9 aa, Artificial Sequence
  synthetic construct; Fab
SGSGSRYAL

SEQ ID NO: 50 - protein, 13 aa, Artificial Sequence
  synthetic construct; Fab
KADYEKHKVYACE

SEQ ID NO: 51 - protein, 9 aa, Artificial Sequence
  synthetic construct; Fab
QQKPGKAPK

SEQ ID NO: 52 - protein, 6 aa, Artificial Sequence
  synthetic construct; Fab
KKVEPK

(19) United States
(12) Patent Application Publication
(10) Pub. No.: US 2014/0288277 A1
(43) Pub. Date: Sep. 25, 2014

(54) Compositions and Methods for Comprising an RNA-Specific Fab Library

(71) Applicant: University of Central Florida Research Foundation, Inc., Orlando, FL (US)

(72) Inventors: Jingdong Ye, Orlando, FL (US); Eileen Sherman, Orlando, FL (US); Jennifer Archer, Oviedo, FL (US)

(21) Appl. No.: 14/213,759

(22) Filed: Mar. 14, 2014

Related U.S. Application Data
(60) Provisional application No. 61/783,906, filed on Mar. 14, 2013.

(51) Int. Cl. C07K 16/44

(52) U.S. Cl. CPC C07K 16/44
USPC 530/387.5; 530/387.3; 435/320.1; 506/14

(57) ABSTRACT

Methods and compositions are provided for studying and targeting RNA structures. In particular, antibodies and antibody fragments that contain an Fab were produced. Phage display libraries that were engineered to produce RNA-specific antibody and antibody fragments were made. Antibodies, or fragments thereof, that have at least one Fab that specifically binds tRNAᵢ^{Met}, the P4-P6 domain of Tetrahymena Group I intron, the glycine riboswitch from Vibrio Cholerae, or the glycine riboswitch from Fusobacterium Nucleatum were identified. The production of these RNA-specific antibodies and antibody fragments can be used to study the structure of the RNA as well as to treat diseases with known RNA targets. For example, an Fab specific to tRNAᵢ^{Met} was produced that can be used to treat cancer in subjects where tRNAᵢ^{Met} is overexpressed.

### CROSS-REFERENCE TO RELATED APPLICATION

This patent application is a nonprovisional patent application claiming priority to and the benefit of the filing date of U.S. Provisional Patent Application Ser. No. 61/783,906, filed Mar. 14, 2013, which is herein incorporated in its entirety.

### REFERENCE TO SEQUENCE LISTING

The Sequence Listing submitted Mar. 14, 2014 as a text file named “26150_{—}0039U2_Sequence_Listing.txt,” created on Mar. 11, 2014, and having a size of 10,498 bytes is hereby incorporated by reference pursuant to 37 C.F.R. §1.52(e)(5).

### FIELD OF THE INVENTION

The disclosed invention is generally in the field of immunology and molecular biology and specifically in the area of antibodies and antibody fragments targeting RNAs.

### BACKGROUND

Non-coding RNAs (ncRNAs) have been recognized as important cancer disease markers and therapeutic targets. Many oncogenic miRNAs (oncomirs)(1,2) and long intergenic ncRNAs (lincRNAs)(3,4) have been shown to promote cell proliferation, and play oncogenic roles in cancer initiation or progression. The ncRNAs often exert their oncogenic functions by interacting with protein partners, making the RNA-protein interfaces important targets for cancer therapy. Targeting the RNA-protein interface with a focus on the oncogenic RNA is more advantageous than targeting its non cancer-specific protein partners, which often have normal cellular functions that could be disrupted and lead to deleterious phenotypes(3). However, currently there is a lack of a robust and general approach in targeting structured RNAs. Such an approach is particularly beneficial to the development of drug-like agents that interfere with cancer-specific RNA-protein interfaces for therapeutic intervention.

Non-coding RNAs have been recognized as novel targets for cancer diagnosis and therapeutics. As RNAs are intrinsically different from proteins in their surface functional groups, secondary and tertiary structures, current methods of targeting ncRNAs are severely limited. Although RNA interferences have been used successfully to target several long ncRNAs in the laboratory setting, targeting them in vivo is very difficult because of their extensive secondary and tertiary structures (13). Furthermore, off-target effects, susceptibility to cellular nuclease activity, potential immunological response, and challenges in cancer-specific delivery of the siRNAs as well as other antisense nucleotide approaches have set major hurdles in developing viable siRNA-based or general antisense-based drugs(13).

Humanized antibodies have been widely and successfully used as therapeutic drugs(11) with annual global sales that totaled 44 billion USD in 2010. Therefore, developing antibodies, particularly humanized antibodies, that target RNA can be beneficial.

What is needed are therapeutic compositions comprising antibodies and antibody fragments that specifically bind RNA, such as antibodies and antibody fragments that target RNAs known to be involved in cancer, such as tRNAᵢ^{Met}. What is also needed are anti-RNA antibody or antibody fragment phage displays for basic RNA biochemical research. Also needed are methods of using RNA-specific antibodies and antibody fragments for treating diseases such as cancer, neurological disorder, and bacterial infections.

### BRIEF SUMMARY

Disclosed are isolated antibodies, or fragments thereof that contain at least one binding site or Fab that specifically binds to tRNAᵢ^{Met}. The antibodies or fragments thereof include complementarity determining regions (CDRs) on the variable regions of the heavy and light chains of the antibody, or fragment thereof, wherein the CDRs of the light chain contain one or more of the amino acid sequences SSRYR (SEQ ID NO:1), YGAYRLSSGVPYR (SEQ ID NO:2), and GSSYPV (SEQ ID NO:3). The antibodies or fragments thereof can also include complementarity determining regions (CDRs) on the variable regions of the heavy and light chains of the antibody, or fragment thereof, wherein the CDRs of the heavy chain comprise one or more of the amino acid sequences NFSGSGI (SEQ ID NO:4), GSGSSRGYTR (SEQ ID NO:5), and SGSGSRYAL (SEQ ID NO:6).

Disclosed are antibodies, or fragments thereof, that bind the P4-P6 domain of Tetrahymena Group I intron, wherein the antibody, or fragment thereof, comprises complementarity determining regions (CDRs) on the variable regions of the heavy and light chains of the antibody, or fragment thereof, wherein the CDRs of the light chain comprise one or more of the amino acid sequences YGYRS (SEQ ID NO:7), YSASGLYRGVPSR (SEQ ID NO:8), and GYRSPV (SEQ ID NO:9). The antibodies or fragments thereof can also include complementarity determining regions (CDRs) on the variable regions of the heavy and light chains of the antibody, or fragment thereof, wherein the CDRs of the heavy chain comprise one or more of the amino acid sequences NLGSGYI (SEQ ID NO:10), SYRPSSGSTR (SEQ ID NO:11), and SYSSRYSYAM (SEQ ID NO:12).

Disclosed are antibodies, or fragments thereof, that bind the P4-P6 domain of Tetrahymena Group I intron, wherein the antibody, or fragment thereof, comprises complementarity determining regions (CDRs) on the variable regions of the heavy and light chains of the antibody, or fragment thereof, wherein the CDRs of the light chain comprise one or more of the amino acid sequences SVSSA (SEQ ID NO:13), YSASSLYSGVPSR (SEQ ID NO:14), and SYSSPI (SEQ ID NO:15). The antibodies or fragments thereof can also include complementarity determining regions (CDRs) on the variable regions of the heavy and light chains of the antibody, or fragment thereof, wherein the CDRs of the heavy chain comprise one or more of the amino acid sequences NLYSSSI (SEQ ID NO:16), SRSPRSGGTS (SEQ ID NO:17), and RAAGMSTYGF (SEQ ID NO:18).

Disclosed are antibodies, or fragments thereof, that bind the glycine riboswitch from Vibrio Cholerae, wherein the antibody, or fragment thereof, comprises complementarity determining regions (CDRs) on the variable regions of the heavy and light chains of the antibody, or fragment thereof, wherein the CDRs of the light chain comprise one or more of the amino acid sequences YSYRS (SEQ ID NO:19), YRASRLYGGVPSR (SEQ ID NO:20), and RSSYPV (SEQ ID NO:21). The antibodies or fragments thereof can also include complementarity determining regions (CDRs) on the variable regions of the heavy and light chains of the antibody, or fragment thereof, wherein the CDRs of the heavy chain comprise one or more of the amino acid sequences NFSGSSI (SEQ ID NO:22), YGSPGYGRTS (SEQ ID NO:23), and SSYGSRSGYAM (SEQ ID NO:24).

Disclosed are antibodies, or fragments thereof, that bind the glycine riboswitch from Vibrio Cholerae, wherein the antibody, or fragment thereof, comprises complementarity determining regions (CDRs) on the variable regions of the heavy and light chains of the antibody, or fragment thereof, wherein the CDRs of the light chain comprise one or more of the amino acid sequences SGSSY (SEQ ID NO:25), YRASSLSSGVPSR (SEQ ID NO:26), and YSSRLL (SEQ ID NO:27). The antibodies or fragments thereof can also include complementarity determining regions (CDRs) on the variable regions of the heavy and light chains of the antibody, or fragment thereof, wherein the CDRs of the heavy chain comprise one or more of the amino acid sequences NIYRYGI (SEQ ID NO:28), YGSPGSGRTY (SEQ ID NO:29), and RYRYRYRSGL (SEQ ID NO:30).

Disclosed are antibodies, or fragments thereof, that bind the glycine riboswitch from Fusobacterium Nucleatum, wherein the antibody, or fragment thereof, comprises complementarity determining regions (CDRs) on the variable regions of the heavy and light chains of the antibody, or fragment thereof, wherein the CDRs of the light chain comprise one or more of the amino acid sequences YSSRY (SEQ ID NO:31), YGAYGLYRGVPYR (SEQ ID NO:32), and SYSYPI (SEQ ID NO:33). The antibodies or fragments thereof can also include complementarity determining regions (CDRs) on the variable regions of the heavy and light chains of the antibody, or fragment thereof, wherein the CDRs of the heavy chain comprise one or more of the amino acid sequences NLGYRYI (SEQ ID NO:34), SGSSGSGSTY(SEQ ID NO:35), and GSSYSYRYAF (SEQ ID NO:36).

The disclosed antibodies, or fragments thereof, can be monoclonal and can be humanized. In one aspect, the antibody fragment can be the Fab portion of an antibody.

Disclosed are bi-specific Fab complexes that contain a therapeutic arm and a delivery arm, wherein the therapeutic arm contains an anti-tRNAᵢ^{Met} Fab. The delivery arm contains an anti-HER2 Fab.

Also disclosed are conjugates that contain any of the disclosed antibodies, or fragments thereof, conjugated to a detection moiety. The detection moiety can be a fluorophore.

Disclosed are pharmaceutical compositions that contain any of the disclosed antibodies, or fragments thereof. Also disclosed are pharmaceutical compositions that contain any of the disclosed Fab complexes.

Disclosed are bacteriophages that contain a nucleic acid encoding any of the disclosed antibodies, or fragments thereof.

Also disclosed are phage display libraries that contain the disclosed bacteriophage. The phage display library can contain phage that express the Fab portion of an antibody, wherein all six CDRs of each Fab are mutated, wherein the mutated CDRs comprise only selectively randomized Tyrosine, Serine, Glycine, and Arginine residues. In one aspect, the CDR do not contain consecutive arginines.

Also disclosed are phage display libraries that contain phage that express the Fab portion of an antibody, wherein all six CDRs of each Fab are mutated, wherein the mutated CDRs comprise only selectively randomized Tyrosine, Serine, Glycine, and Arginine residues, wherein two separate degenerate codons are applied in an alternating fashion in a consecutive CDR sequence, wherein one degenerate codon comprises the nucleic acid sequence TMT, wherein M is A or C, and equal portions of Tyr and Ser are encoded, wherein the other degenerate codon comprises VGT, wherein V is A, C, or G, and equal portions of Ser, Gly, and Arg are encoded.

Disclosed are phage display libraries, wherein the Fab produced by the phage specifically bind RNA.

Also disclosed are kits containing the disclosed phage and phage display libraries.

Disclosed are methods of treating cancer including administering to a subject in need thereof any of the disclosed pharmaceutical compositions. The cancer can be breast cancer or pancreatic cancer. The administration of the pharmaceutical composition can be to a subject that overexpresses tRNAᵢ^{Met}.

Disclosed are methods of treating cancer including administering the disclosed Fab complexes, wherein the therapeutic arm comprises tRNAᵢ^{Met} Fab and the delivery arm comprises an anti-HER2 Fab.

Disclosed are methods of treating cancer including administering the disclosed Fab complexes, wherein the therapeutic arm comprises tRNAᵢ^{Met} Fab, wherein the anti-tRNAᵢ^{Met} Fab inhibits cell proliferation, blocks binding of tRNAᵢ^{Met} with elongation factors, or prevents tRNAᵢ^{Met} from initiating polypeptide synthesis.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1 is a ribbon diagram exemplifying antibody cancer therapy targeting tRNAᵢ^{Met}. Fab-tRNA binding may block posttranscriptional modification, amino acid charging and/or ribosomal binding.

FIG. 2 is a schematic representation of chemically crosslinked bispecific antibodies. Figure adapted from Chames and Baty, mAbs (2009), 1, 539-547.

FIGS. 3A, 3B and 3C show a YSGR Minimal Codon Fab Library. A) Fab fragments are portions of an IgG antibody, diagrammed here, used as chaperones to bind and stabilize their target RNAs. The six CDRs are orange, called L1-3 and H1-3 (light chain, heavy chain). B) Randomized Fab library was constructed through a Kunkel mutagenesis reaction, annealing a total of 16 mutagenic primers amongst the template sequence's six CDR regions. The left lane in this 1% agarose gel is a standard 1 kb ladder purchased from NEB, while the right is the Kunkel mutagenized template. C) The YSGR Minimal codon Library of Fab Fragments consists of unique Fabs that are randomized in CDRs with tyrosine, serine, glycine, and arginine. Some ‘scaffold’ residues were chosen to be conserved in all Fabs, in order to not disrupt the protein's native structural integrity. The Kabat numbering system was used to locate residues within the Fab amino acid sequence. Shown here is the codon design for one of the six CDRs, L2. Blue are unaltered flanking DNA sequences, red are unaltered ‘scaffold’ residues, and randomized YSGR residues are black. As can be seen, two alternating codons were designed for each CDR, with the exception of H3, which had 6, as there were three pairs of different lengths.

FIGS. 4A, 4B, 4C, and 4D show RNA Targets for YSGRMin Library. To test the YSGR minimal codon library three RNA targets were chosen: P4P6, FNIII, VCLD1. RNA targets were synthesized via the following methods: PCR, EcoRI and HindIII digestion, ligation into digested pUC19 vector, heat shock transformation into competent JM109 cells, Qiagen miniprep kit, small scale EarI digestion, sequence confirmation by Genewiz, Qiagen midiprep kit, PCA extraction, ethanol precipitation, large scale EarI digestion, PCA extraction, ethanol precipitation, in vitro transcription, buffer exchange into 2 mM Sodium Citrate, PAGE purification, electroelution in TE Buffer pH 8, PCA extraction, ethanol precipitation. A) VC and B) FN riboswitch secondary structures at top, C)P4P6 tertiary structure below in yellow. D) 2% agarose gels run at 100V for 45 min., confirming PCR reaction for first step in P4P6tag synthesis; EarI digestion reaction after DNA sequencing, before transcription; and confirming in vitro transcription reaction.

FIGS. 5A and 5B shows the initial Screening of Library. A) Phage selection followed by ELISA screened YSGRMin for RNA-binding Fabs. Selected specific Fabs were sequenced by Genewiz. 16 Fabs of interest were expressed as soluble proteins. B) Selection results. C) DNA sequencing results, light and heavy chain CDRs shown.

FIG. 6: EMSA Specificity Check of 16 Selected Fabs. After expression and purification, an EMSA native gel was run on each Fab in complex with its RNA target. As can be seen, 5 Fabs shift, so these are specific binders.

FIG. 7 shows the Dot Blot Binding Affinity of EMSA Specific Binding Fabs. After EMSA identification as target specific binders, Fabs' binding affinity for their radiolabeled RNA target was determined by dot blot assay. Above is one exemplar graph obtained, followed by a table of all values obtained.

FIG. 8 is a schematic of the selection of the anti-tRNAᵢ^{Met} Fab.

FIG. 9 is a flow chart of the synthesis of elongator tRNA^{Met} as an example for RNA preparation.

FIG. 10 shows the production of Fab fragments (particular, anti-Her2 Fab) and the binding assays to determine Kd of novel anti-tRNAi^{Met} Fab to tRNAi^{Met} versus elongator tRNA^{Met}.

FIG. 11 shows a 2% native agarose gel with 4 lanes. In Lane 1 (left to right), it contains a 100 kb DNA ladder. Lane 2 contains the EarI digested DNA of elongator tRNA. Lane 3 and 4 contain two different concentrations of elongator tRNA post transcription. The bright bands show a successful transcription, and Lane 4 shows pure RNA.

FIG. 12 shows from left to right. SDS-PAGE—Lane 3 shows a 50 kD band which signifies accumulation of anti-tRNAiMet—signifying successful construction and purification. Lanes 4 and 5 show 100 kD bands that are dimers of anti-HER2 Fab in non-reducing conditions. These two lanes signify successful construction of the anti-HER2 Fab as confirmed by gel electrophoresis and sequencing.

FIG. 13 shows an 8% native PAGE. From Left to Right—The Lanes contain: 1: elongator tRNA only 2: tRNAe+1.5 eq anti-tRNAᵢ^{Met}. 3: elongator tRNA+2.0 eq anti-tRNAᵢ^{Met}. 4: tRNAi only. 5: tRNAᵢ+1.5 eq anti-tRNAᵢ^{Met}. 6: tRNAᵢ+2.0 eq anti-tRNAᵢ^{Met}. 7: Marker dye (native gel loading).

FIG. 14 shows the characterization of the binding affinity and specificity of Fab7. In particular, a filter binding assay of Fab7 binding to tRNAᵢ^{Met} was performed.

FIG. 15 is a line graph showing the effect of the Fabs on in vitro GFP expression using 1-step human in vitro protein expression kit.

FIG. 16 shows the stepwise construction of bispecific Fabs.

FIG. 17 demonstrates that upon entry of 1.5 μM Fab 7 into SK-BR-3 breast cancer cells using 25 μM Chariot™, Cell viability was reduced by 52% compared to the mock (1×PBS) treated control where the 25 μM only Chariot™ control only reduced viability by 19%, a 33% difference. The cell morphology appeared normal by observing in an inverted microscope suggesting a cytostatic effect. A biologic that reduces protein expression would not be expected to be cytotoxic and instead would retard growth which is desirable. There is a growing demand for cytostatic drugs which reduce unintended side-effects.

### DETAILED DESCRIPTION

The disclosed method and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

### A. Definitions

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

“Antibody, or fragment thereof,” refers to a whole antibody or an antibody fragment. The antibody fragment includes all fragments that have at least one Fab portion. For example, an antibody fragment can be an Fab fragment.

The phrase “complementarity determining region” or “CDR” refer to regions within the Fab portion of an antibody that determine the specificity and affinity for binding to specific antigens. The CDR is found in the variable region of the Fab. Three CDRs (L1, L2, L3) are present in the variable region of the light chain and three CDRs (H1, H2, H3) are present in the variable region of the heavy chain.

As used herein the term “effective amount” or “therapeutically effective amount” means a dosage sufficient to treat, inhibit, or alleviate one or more symptoms of the disease or disorder being treated or to otherwise provide a desired pharmacologic and/or physiologic effect. The precise dosage will vary according to a variety of factors such as subject-dependent variables (e.g., age, immune system health, etc.), the disease, and the treatment being effected. Therapeutically effective amounts of tRNAᵢ^{Met}-specific Fab complexes inhibit or prevent initiation of peptide synthesis

The term “Fab” refers to the antigen binding fragment of an antibody. The Fab portion of an antibody can contain one light and one heavy chain and the light chain and heavy chain can each contain a variable and a constant region. The variable regions contain the antigen-binding sites.

The term “Fab fragment” refers to an antibody fragment that contains the Fab region of the antibody. Fab fragments can contain only the Fab region of an antibody. In some instances, the Fab fragment can contain the Fab region of an antibody in addition to part of the constant region (Fc) of an antibody.

“Phage display” or “phage display library” is a pool of bacteriophage that each contains a nucleic acid sequence that encodes a different protein. The bacteriophage can then be used to study the interactions of different proteins or nucleic acids with the proteins encoded by the bacteriophage.

The phrases “tRNAᵢ^{Met}-specific antibodies, or fragments thereof” and “antibodies, or fragments thereof, that specifically bind tRNAᵢ^{Met}” can be used interchangeably. They both refer to an antibody, or fragment thereof, that specifically binds tRNAᵢ^{Met}.

It must be noted that as used herein and in the appended claims, the singular forms “a”, “an”, and “the” include plural reference unless the context clearly dictates otherwise. Thus, for example, reference to “a antibody” includes a plurality of such antibodies, reference to “the Fab” is a reference to one or more Fab fragments and equivalents thereof known to those skilled in the art, and so forth.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

Those skilled in the art will recognize, or be able to ascertain using no more than routine experimentation, many equivalents to the specific embodiments of the method and compositions described herein. Such equivalents are intended to be encompassed by the following claims.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### B. Antibodies and Antibody Fragments

Disclosed are antibodies, or fragments thereof, comprising at least one Fab that specifically binds tRNAᵢ^{Met}, the P4-P6 domain of Tetrahymena Group I intron, the glycine riboswitch from Vibrio Cholerae, or the glycine riboswitch from Fusobacterium Nucleatum.

The disclosed antibodies and antibody fragments can be monoclonal. In one aspect, the antibodies and antibody fragments can be humanized. In another aspect, the antibodies can be IgG antibodies.

1. Antibodies and Antibody Fragments that Bind tRNAᵢ^{Met}

Disclosed herein is an isolated antibody, or fragment thereof, comprising at least one binding site or Fab that specifically binds to tRNAiMet. In one aspect, the tRNAiMet can be vertebrate tRNAiMet.

The disclosed antibodies, or fragments thereof, that specifically bind tRNAᵢ^{Met} can have CDRs on the variable regions of the heavy and light chains of the Fab, wherein the CDRs of the light chain comprise one or more of the amino acid sequences SSRYR (SEQ ID NO:1), YGAYRLSSGVPYR (SEQ ID NO:2), and GSSYPV (SEQ ID NO:3). Further disclosed are tRNAᵢ^{Met}-specific antibodies, or fragments thereof, that comprise one or more of the amino acid sequences NFSGSGI (SEQ ID NO:4), GSGSSRGYTR (SEQ ID NO:5), and SGSGSRYAL (SEQ ID NO:6) on the CDRs of the heavy chain.

The disclosed antibodies or antibody fragments that bind tRNAᵢ^{Met} can contain one or more of the disclosed CDR sequences for the heavy and light chains or a combination thereof. For example, the encoded Fab fragment can have one, two, or all three of the amino acid sequences SSRYR (SEQ ID NO:1), YGAYRLSSGVPYR (SEQ ID NO:2), and GSSYPV (SEQ ID NO:3) in the CDR of the light chain. In addition to having one, two, or all three of the CDR light chain sequences, the Fab fragment can also have one, two or all three of the amino acid sequences NFSGSGI (SEQ ID NO:4), GSGSSRGYTR (SEQ ID NO:5), and SGSGSRYAL (SEQ ID NO:6) in the CDR of the heavy chain.

The disclosed isolated antibody, or fragment thereof comprising at least one Fab that specifically binds to vertebrate tRNAᵢ^{Met} can be a monoclonal antibody and in some instances can be humanized. In one aspect, the antibody fragment can be the Fab portion of an antibody, also called a Fab fragment. The Fab portion of an antibody can be a Fab portion of an IgG antibody.

2. Antibodies and Antibody Fragments that Bind P4-P6 Domain of Tetrahymena Group I Intron

The disclosed antibodies, or fragments thereof, that specifically bind the P4-P6 domain of Tetrahymena Group I intron can have CDRs on the variable regions of the heavy and light chains of the Fab, wherein the CDRs of the light chain comprise one or more of the amino acid sequences YGYRS (SEQ ID NO:7), YSASGLYRGVPSR (SEQ ID NO:8), and GYRSPV (SEQ ID NO:9). Further disclosed are antibodies, or fragments thereof, that bind P4-P6 domain of Tetrahymena Group I intron that comprise one or more of the amino acid sequences NLGSGYI (SEQ ID NO:10), SYRPSSGSTR (SEQ ID NO:11), and SYSSRYSYAM (SEQ ID NO:12) on the CDRs of the heavy chain.

The disclosed antibodies or antibody fragments that specifically bind the P4-P6 domain of Tetrahymena Group I intron can contain one or more of the disclosed CDR sequences for the heavy and light chains or a combination thereof. For example, the encoded Fab fragment can have one, two, or all three of the amino acid sequences YGYRS (SEQ ID NO:7), YSASGLYRGVPSR (SEQ ID NO:8), and GYRSPV (SEQ ID NO:9) in the CDR of the light chain. In addition to having one, two, or all three of the CDR light chain sequence, the Fab fragment can also have one, two or all three of the amino acid sequences NLGSGYI (SEQ ID NO:10), SYRPSSGSTR (SEQ ID NO:11), and SYSSRYSYAM (SEQ ID NO:12) in the CDR of the heavy chain.

The disclosed antibodies, or fragments thereof, that specifically bind the P4-P6 domain of Tetrahymena Group I intron can have CDRs on the variable regions of the heavy and light chains of the Fab, wherein the CDRs of the light chain comprise one or more of the amino acid sequences SVSSA (SEQ ID NO:13), YSASSLYSGVPSR (SEQ ID NO:14), and SYSSPI (SEQ ID NO:15). Further disclosed are antibodies, or fragments thereof, that bind P4-P6 domain of Tetrahymena Group I intron that comprise one or more of the amino acid sequences NLYSSSI (SEQ ID NO:16), SRSPRSGGTS (SEQ ID NO:17), and RAAGMSTYGF (SEQ ID NO:18) on the CDRs of the heavy chain.

The disclosed antibodies or antibody fragments that specifically bind the P4-P6 domain of Tetrahymena Group I intron can contain one or more of the disclosed CDR sequences for the heavy and light chains or a combination thereof. For example, the encoded Fab fragment can have one, two, or all three of the amino acid sequences SVSSA (SEQ ID NO:13), YSASSLYSGVPSR (SEQ ID NO:14), and SYSSPI (SEQ ID NO:15) in the CDR of the light chain. In addition to having one, two, or all three of the CDR light chain sequence, the Fab fragment can also have one, two or all three of the amino acid sequences NLYSSSI (SEQ ID NO:16), SRSPRSGGTS (SEQ ID NO:17), and RAAGMSTYGF (SEQ ID NO:18) in the CDR of the heavy chain.

3. Antibodies and Antibody Fragments that Bind Glycine Riboswitch from Vibrio Cholerae

The disclosed antibodies, or fragments thereof, that specifically bind the glycine riboswitch from Vibrio Cholerae can have CDRs on the variable regions of the heavy and light chains of the Fab, wherein the CDRs of the light chain comprise one or more of the amino acid sequences YSYRS (SEQ ID NO:19), YRASRLYGGVPSR (SEQ ID NO:20), and RSSYPV (SEQ ID NO:21). Further disclosed are antibodies, or fragments thereof, that bind the glycine riboswitch from Vibrio Cholerae that comprise one or more of the amino acid sequences NFSGSSI (SEQ ID NO:22), YGSPGYGRTS (SEQ ID NO:23), and SSYGSRSGYAM (SEQ ID NO:24) on the CDRs of the heavy chain.

The disclosed antibodies or antibody fragments that specifically bind the glycine riboswitch from Vibrio Cholerae can contain one or more of the disclosed CDR sequences for the heavy and light chains or a combination thereof. For example, the encoded Fab fragment can have one, two, or all three of the amino acid sequences YSYRS (SEQ ID NO:19), YRASRLYGGVPSR (SEQ ID NO:20), and RSSYPV (SEQ ID NO:21) in the CDR of the light chain. In addition to having one, two, or all three of the CDR light chain sequence, the Fab fragment can also have one, two or all three of the amino acid sequences NFSGSSI (SEQ ID NO:22), YGSPGYGRTS (SEQ ID NO:23), and SSYGSRSGYAM (SEQ ID NO:24) in the CDR of the heavy chain.

The disclosed antibodies, or fragments thereof, that specifically bind the glycine riboswitch from Vibrio Cholerae can have CDRs on the variable regions of the heavy and light chains of the Fab, wherein the CDRs of the light chain comprise one or more of the amino acid sequences SGSSY (SEQ ID NO:25), YRASSLSSGVPSR (SEQ ID NO:26), and YSSRLL (SEQ ID NO:27). Further disclosed are antibodies, or fragments thereof, that bind the glycine riboswitch from Vibrio Cholerae that comprise one or more of the amino acid sequences NIYRYGI (SEQ ID NO:28), YGSPGSGRTY (SEQ ID NO:29), and RYRYRYRSGL (SEQ ID NO:30) on the CDRs of the heavy chain.

The disclosed antibodies or antibody fragments that specifically bind the glycine riboswitch from Vibrio Cholerae can contain one or more of the disclosed CDR sequences for the heavy and light chains or a combination thereof. For example, the encoded Fab fragment can have one, two, or all three of the amino acid sequences SGSSY (SEQ ID NO:25), YRASSLSSGVPSR (SEQ ID NO:26), and YSSRLL (SEQ ID NO:27) in the CDR of the light chain. In addition to having one, two, or all three of the CDR light chain sequence, the Fab fragment can also have one, two or all three of the amino acid sequences NIYRYGI (SEQ ID NO:28), YGSPGSGRTY (SEQ ID NO:29), and RYRYRYRSGL (SEQ ID NO:30) in the CDR of the heavy chain.

4. Antibodies and Antibody Fragments that Bind Glycine Riboswitch from Fusobacterium nucleatum

The disclosed antibodies, or fragments thereof, that specifically bind the glycine riboswitch from Fusobacterium Nucleatum can have CDRs on the variable regions of the heavy and light chains of the Fab, wherein the CDRs of the light chain comprise one or more of the amino acid sequences YSSRY (SEQ ID NO:31), YGAYGLYRGVPYR (SEQ ID NO:32), and SYSYPI (SEQ ID NO:33). Further disclosed are antibodies, or fragments thereof, that bind the glycine riboswitch from Fusobacterium Nucleatum that comprise one or more of the amino acid sequences NLGYRYI (SEQ ID NO:34), SGSSGSGSTY(SEQ ID NO:35), and GSSYSYRYAF (SEQ ID NO:36) on the CDRs of the heavy chain.

The disclosed antibodies or antibody fragments that specifically bind the glycine riboswitch from Fusobacterium Nucleatum can contain one or more of the disclosed CDR sequences for the heavy and light chains or a combination thereof. For example, the encoded Fab fragment can have one, two, or all three of the amino acid sequences YSSRY (SEQ ID NO:31), YGAYGLYRGVPYR (SEQ ID NO:32), and SYSYPI (SEQ ID NO:33) in the CDR of the light chain. In addition to having one, two, or all three of the CDR light chain sequence, the Fab fragment can also have one, two or all three of the amino acid sequences NLGYRYI (SEQ ID NO:34), SGSSGSGSTY(SEQ ID NO:35), and GSSYSYRYAF (SEQ ID NO:36) in the CDR of the heavy chain.

The disclosed antibodies or antibody fragments that specifically bind the glycine riboswitch from Fusobacterium nucleatum can contain one or more of the disclosed CDR sequences for the heavy and light chains or a combination thereof. For example, the encoded Fab fragment can have one, two, or all three of the amino acid sequences YSSRY (SEQ ID NO:31), YGAYGLYRGVPYR (SEQ ID NO:32), and SYSYPI (SEQ ID NO:33) in the CDR of the light chain. In addition to having one, two, or all three of the CDR light chain sequence, the Fab fragment can also have one, two or all three of the amino acid sequences NLGYRYI (SEQ ID NO:34), SGSSGSGSTY(SEQ ID NO:35), and GSSYSYRYAF (SEQ ID NO:36) in the CDR of the heavy chain.

5. Antibodies Generally

The term “antibodies” is used herein in a broad sense and includes both polyclonal and monoclonal antibodies. Human or humanized versions of antibodies, or fragments thereof, are also disclosed. Antibody and antibody fragments also includes, chimeric antibodies and hybrid antibodies, and fragments, such as F(ab′)2, Fab′, Fab and the like, including hybrid fragments Thus, fragments of the antibodies that retain the ability to bind their specific antigens are provided. Such antibodies and fragments can be made by techniques known in the art and can be screened for specificity and activity according to the methods set forth in the Examples and in general methods for producing antibodies and screening antibodies for specificity and activity (See Harlow and Lane. Antibodies, A Laboratory Manual. Cold Spring Harbor Publications, New York, (1988)).

Also included within the meaning of “antibody or fragments thereof” are conjugates of antibody fragments and antigen binding proteins (single chain antibodies) as described, for example, in U.S. Pat. No. 4,704,692, the contents of which are hereby incorporated by reference.

As used herein, the term “antibody” or “antibody fragment” can also refer to a human antibody and/or a humanized antibody or antibody fragment. Many non-human antibodies (e.g., those derived from mice, rats, or rabbits) are naturally antigenic in humans, and thus can give rise to undesirable immune responses when administered to humans. Therefore, the use of human or humanized antibodies or antibody fragments in the methods of the invention serves to lessen the chance that an antibody or antibody fragment administered to a human will evoke an undesirable immune response

i. Monoclonal Antibodies

The term “monoclonal antibody” as used herein refers to an antibody obtained from a substantially homogeneous population of antibodies, i.e., the individual antibodies within the population are identical except for possible naturally occurring mutations that can be present in a small subset of the antibody molecules. The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, as long as they exhibit the desired antagonistic activity (See, U.S. Pat. No. 4,816,567 and Morrison et al., Proc. Natl. Acad. Sci. USA, 81:6851-6855 (1984)).

The disclosed monoclonal antibodies can be made using any procedure which produces monoclonal antibodies. For example, monoclonal antibodies of the invention can be prepared using hybridoma methods, such as those described by Kohler and Milstein, Nature, 256:495 (1975). In a hybridoma method, a mouse or other appropriate host animal is typically immunized with an immunizing agent to elicit lymphocytes that produce or are capable of producing antibodies that will specifically bind to the immunizing agent. Alternatively, the lymphocytes can be immunized in vitro, e.g., using the binding domains of the compositions described, herein, such as the ligand binding domain, described herein.

The monoclonal antibodies can also be made by recombinant DNA methods, such as those described in U.S. Pat. No. 4,816,567 (Cabilly et al.). DNA encoding the disclosed monoclonal antibodies can be readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as simian COS cells, Chinese hamster ovary (CHO) cells, plasmacytoma cells, or myeloma cells that do not otherwise produce immunoglobulin protein, to obtain the synthesis of monoclonal antibodies in the recombinant host cells. The DNA also may be modified, for example, by substituting the coding sequence for human heavy and light chain constant domains in place of the homologous murine sequences (U.S. Pat. No. 4,816,567) or by covalently joining to the immunoglobulin coding sequence all or part of the coding sequence for a non-immunoglobulin polypeptide. Libraries of antibodies or active antibody fragments can also be generated and screened using phage display techniques, e.g., as described in U.S. Pat. No. 5,804,440 to Burton et al. and U.S. Pat. No. 6,096,441 to Barbas et al.

In vitro methods are also suitable for preparing monovalent antibodies. Digestion of antibodies to produce fragments thereof, particularly, Fab fragments, can be accomplished using routine techniques known in the art. For instance, digestion can be performed using papain. Examples of papain digestion are described in WO 94/29348 published Dec. 22, 1994 and U.S. Pat. No. 4,342,566. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen.

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment can be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment. (Zoller, M. J. Curr. Opin. Biotechnol. 3:348-354, 1992).

ii. Human Antibodies

The human antibodies of the invention can be prepared using any technique. Examples of techniques for human monoclonal antibody production include those described by Cole et al. (Monoclonal Antibodies and Cancer Therapy, Alan R. Liss, p. 77, 1985) and by Boerner et al. (J. Immunol., 147(1):86-95, 1991). Human antibodies of the invention (and fragments thereof) can also be produced using phage display libraries (Hoogenboom et al., J. Mol. Biol., 227:381, 1991; Marks et al., J. Mol. Biol., 222:581, 1991).

The human antibodies of the invention can also be obtained from transgenic animals. For example, transgenic, mutant mice that are capable of producing a full repertoire of human antibodies, in response to immunization, have been described (see, e.g., Jakobovits et al., Proc. Natl. Acad. Sci. USA, 90:2551-255 (1993); Jakobovits et al., Nature, 362:255-258 (1993); Bruggermann et al., Year in Immunol., 7:33 (1993)). Specifically, the homozygous deletion of the antibody heavy chain joining region (J(H)) gene in these chimeric and germ-line mutant mice results in complete inhibition of endogenous antibody production, and the successful transfer of the human germ-line antibody gene array into such germ-line mutant mice results in the production of human antibodies upon antigen challenge. Antibodies having the desired activity are selected using Env-CD4-co-receptor complexes as described herein.

iii. Humanized Antibodies

The disclosed antibodies and antibody fragments can be generated in other species and “humanized” for administration in humans. Humanized forms of non-human (e.g., murine) antibodies are chimeric immunoglobulins, immunoglobulin chains or fragments thereof (such as Fv, Fab, Fab′, F(ab′)2, or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues that are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin (Jones et al., Nature, 321:522-525 (1986); Riechmann et al., Nature, 332:323-327 (1988); and Presta, Curr. Op. Struct. Biol., 2:593-596 (1992)).

Methods for humanizing non-human antibodies are well known in the art. Generally, a humanized antibody has one or more amino acid residues introduced into it from a source that is non-human. These non-human amino acid residues are often referred to as “import” residues, which are typically taken from an “import” variable domain. Humanization can be essentially performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The choice of human variable domains, both light and heavy, to be used in making the humanized antibodies is very important in order to reduce antigenicity. According to the “best-fit” method, the sequence of the variable domain of a rodent antibody is screened against the entire library of known human variable domain sequences. The human sequence which is closest to that of the rodent is then accepted as the human framework (FR) for the humanized antibody (Sims et al., J. Immunol., 151:2296 (1993) and Chothia et al., J. Mol. Biol., 196:901 (1987)). Another method uses a particular framework derived from the consensus sequence of all human antibodies of a particular subgroup of light or heavy chains. The same framework may be used for several different humanized antibodies (Carter et al., Proc. Natl. Acad. Sci. USA, 89:4285 (1992); Presta et al., J. Immunol., 151:2623 (1993)).

It is further important that antibodies be humanized with retention of high affinity for the antigen and other favorable biological properties. To achieve this goal, according to a preferred method, humanized antibodies are prepared by a process of analysis of the parental sequences and various conceptual humanized products using three dimensional models of the parental and humanized sequences. Three dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the consensus and import sequence so that the desired antibody characteristic, such as increased affinity for the target antigen(s), is achieved. In general, the CDR residues are directly and most substantially involved in influencing antigen binding (see, WO 94/04679, published 3 Mar. 1994).

### C. Fab Complexes and Conjugates

Disclosed are Fab complexes and conjugates that contain at least one of the disclosed Fab fragments. The Fab complexes can contain two or more Fab fragments. The conjugates can include the disclosed antibodies or antibody fragments conjugated to a detection moiety.

Disclosed are bi-specific Fab complexes comprising a therapeutic arm and a delivery arm, wherein the therapeutic arm comprises an anti-tRNAᵢ^{Met} Fab. The therapeutic arm, or anti-tRNAᵢ^{Met} Fab, is the component of the bi-specific Fab complex that provides a therapeutic effect. The therapeutic effect can be a decrease in activity of the Fab target. For example, the therapeutic effect of anti-tRNAᵢ^{Met} Fab can be a decrease or increase in the downstream effects of tRNAᵢ^{Met}. In one aspect, the therapeutic effect can be a decrease or inhibition of polypeptide synthesis.

Disclosed are bi-specific Fab complexes comprising a therapeutic arm and a delivery arm, wherein the therapeutic arm comprises an anti-tRNAiMet Fab and the delivery arm comprises an anti-HER2 Fab. The delivery arm of the bi-specific Fab complex is responsible for delivering or targeting the therapeutic arm to the specific area of interest. Thus, using anti-HER2 Fab as the delivery arm allows for translocation of the Fab complex into the cancer cell wherein the therapeutic arm can exert its effects.

In one aspect, the Fab complex contains more than one therapeutic arm.

Disclosed are conjugates wherein the disclosed antibodies or antibody fragments are conjugated to a detection moiety. The detection moiety can be a fluorophore. The detection moiety can be an enzyme, biotin, metal, or epitope tag. Other known or newly discovered detectable markers are contemplated for use with the provided conjugates. The detectable agent can be one or more imaging agents, including but not limited to radiologic contrast agents or fluorescing imaging agents.

The conjugates can be coupled or linked to the detection moiety using known techniques in the art. The antibody or antibody fragment can be directly or indirectly coupled to the detection moiety. The antibody or antibody fragment can be covalently or non-covalently coupled to the detection moiety.

### D. Pharmaceutical Compositions

Disclosed are pharmaceutical compositions comprising the disclosed antibodies and antibody fragments. Also disclosed are pharmaceutical compositions comprising the disclosed Fab complexes.

Pharmaceutical compositions can be administered to a subject in need thereof, such as a patient with an over expression of tRNAᵢ^{Met}, using methods that are known in the art. Pharmaceutical compositions are typically administered in an effective amount to prevent or reduce one or more symptoms in patients with an over expression of tRNAᵢ^{Met}, for example, proliferation of cancer cells.

The disclosed pharmaceutical compositions may be for administration by parenteral (intramuscular, intraperitoneal, intravenous (IV) or subcutaneous injection), transdermal (either passively or using iontophoresis or electroporation), or transmucosal (nasal, vaginal, rectal, or sublingual) routes of administration. The compositions may also be administered using bioerodible inserts and may be delivered directly to an appropriate lymphoid tissue (e.g., spleen, lymph node, or mucosal-associated lymphoid tissue) or directly to an organ or tumor. The compositions can be formulated in dosage forms appropriate for each route of administration.

1. Pharmaceutically Acceptable Carriers

The compositions disclosed herein can be used prophylactically and therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers can be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions can include carriers, thickeners, diluents, buffers, preservatives, surface active agents and the like in addition to the molecule of choice. Pharmaceutical compositions can also include one or more active ingredients such as antimicrobial agents, antiinflammatory agents, anesthetics, and the like.

The pharmaceutical composition can be administered in a number of ways depending on whether local or systemic treatment is desired, and on the area to be treated. Administration can be topically (including ophthalmically, vaginally, rectally, intranasally), orally, by inhalation, or parenterally, for example by intravenous drip, subcutaneous, intraperitoneal or intramuscular injection. The disclosed antibodies and antibody fragments can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives can also be present such as, for example, antimicrobials, anti-oxidants, chelating agents, and inert gases and the like.

Formulations for topical administration can include ointments, lotions, creams, gels, drops, suppositories, sprays, liquids and powders. Conventional pharmaceutical carriers, aqueous, powder or oily bases, thickeners and the like may be necessary or desirable. Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions can be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

i. Liposomes

Pharmaceutical composition can be carried in a liposome. Liposomes can be used to package any biologically active agent for delivery to cells. Materials and procedures for forming liposomes are well-known to those skilled in the art. Upon dispersion in an appropriate medium, a wide variety of phospholipids swell, hydrate and form multilamellar concentric bilayer vesicles with layers of aqueous media separating the lipid bilayers. These systems are referred to as multilamellar liposomes or multilamellar lipid vesicles (“MLVs”) and have diameters within the range of 10 nm to 100 .mu.m. These MLVs were first described by Bangham, et al., J. Mol. Biol. 13:238-252 (1965). In general, lipids or lipophilic substances are dissolved in an organic solvent. When the solvent is removed, such as under vacuum by rotary evaporation, the lipid residue forms a film on the wall of the container. An aqueous solution that typically contains electrolytes or hydrophilic biologically active materials is then added to the film. Large MLVs are produced upon agitation. When smaller MLVs are desired, the larger vesicles are subjected to sonication, sequential filtration through filters with decreasing pore size or reduced by other forms of mechanical shearing. There are also techniques by which MLVs can be reduced both in size and in number of lamellae, for example, by pressurized extrusion (Barenholz, et al., FEBS Lett. 99:210-214 (1979)).

Liposomes can also take the form of unilamnellar vesicles, which are prepared by more extensive sonication of MLVs, and are made of a single spherical lipid bilayer surrounding an aqueous solution. Unilamellar vesicles (“ULVs”) can be small, having diameters within the range of 20 to 200 nm, while larger ULVs can have diameters within the range of 200 nm to 2 .mu.m. There are several well-known techniques for making unilamellar vesicles. In Papahadjopoulos, et al., Biochim et Biophys Acta 135:624-238 (1968), sonication of an aqueous dispersion of phospholipids produces small ULVs having a lipid bilayer surrounding an aqueous solution. Schneider, U.S. Pat. No. 4,089,801 describes the formation of liposome precursors by ultrasonication, followed by the addition of an aqueous medium containing amphiphilic compounds and centrifugation to form a biomolecular lipid layer system.

Small ULVs can also be prepared by the ethanol injection technique described by Batzri, et al., Biochim et Biophys Acta 298:1015-1019 (1973) and the ether injection technique of Deamer, et al., Biochim et Biophys Acta 443:629-634 (1976). These methods involve the rapid injection of an organic solution of lipids into a buffer solution, which results in the rapid formation of unilamellar liposomes. Another technique for making ULVs is taught by Weder, et al. in “Liposome Technology”, ed. G. Gregoriadis, CRC Press Inc., Boca Raton, Fla., Vol. I, Chapter 7, pg. 79-107 (1984). This detergent removal method involves solubilizing the lipids and additives with detergents by agitation or sonication to produce the desired vesicles.

Papahadjopoulos, et al., U.S. Pat. No. 4,235,871, describes the preparation of large ULVs by a reverse phase evaporation technique that involves the formation of a water-in-oil emulsion of lipids in an organic solvent and the drug to be encapsulated in an aqueous buffer solution. The organic solvent is removed under pressure to yield a mixture which, upon agitation or dispersion in an aqueous media, is converted to large ULVs. Suzuki et al., U.S. Pat. No. 4,016,100, describes another method of encapsulating agents in unilamellar vesicles by freezing/thawing an aqueous phospholipid dispersion of the agent and lipids.

In addition to the MLVs and ULVs, liposomes can also be multivesicular. Described in Kim, et al., Biochim et Biophys Acta 728:339-348 (1983), these multivesicular liposomes are spherical and contain internal granular structures. The outer membrane is a lipid bilayer and the internal region contains small compartments separated by bilayer septum. Still yet another type of liposomes are oligolamellar vesicles (“OLVs”), which have a large center compartment surrounded by several peripheral lipid layers. These vesicles, having a diameter of 2-15 .mu.m, are described in Callo, et al., Cryobiology 22(3):251-267 (1985).

Mezei, et al., U.S. Pat. Nos. 4,485,054 and 4,761,288 also describe methods of preparing lipid vesicles, More recently, Hsu, U.S. Pat. No. 5,653,996 describes a method of preparing liposomes utilizing aerosolization and Yiournas, et al., U.S. Pat. No. 5,013,497 describes a method for preparing liposomes utilizing a high velocity-shear mixing chamber. Methods are also described that use specific starting materials to produce ULVs (Wallach, et al., U.S. Pat. No. 4,853,228) or OLVs (Wallach, U.S. Pat. Nos. 5,474,848 and 5,628,936).

A comprehensive review of all the aforementioned lipid vesicles and methods for their preparation are described in “Liposome Technology”, ed. G. Gregoriadis, CRC Press Inc., Boca Raton, Fla., Vol. I, II & III (1984). This and the aforementioned references describing various lipid vesicles suitable for use in the invention are incorporated herein by reference.

Fatty acids (i.e., lipids) that can be conjugated to the provided compositions include those that allow the efficient incorporation of the disclosed compositions into liposomes. Generally, the fatty acid is a polar lipid. Thus, the fatty acid can be a phospholipid. The provided compositions can include either natural or synthetic phospholipid. The phospholipids can be selected from phospholipids containing saturated or unsaturated mono or disubstituted fatty acids and combinations thereof. The phospholipids can also be synthetic. Synthetic phospholipids are readily available commercially from various sources, such as AVANTI Polar Lipids (Albaster, Ala.); Sigma Chemical Company (St. Louis, Mo.). These synthetic compounds may be varied and may have variations in their fatty acid side chains not found in naturally occurring phospholipids. The fatty acid can have unsaturated fatty acid side chains with C14, C16, C18 or C20 chains length in either or both the PS or PC. Synthetic phospholipids can have dioleoyl (18:1)-PS; palmitoyl (16:0)-oleoyl (18:1)-PS, dimyristoyl (14:0)-PS; dipalmitoleoyl (16:1)-PC, dipalmitoyl (16:0)-PC, dioleoyl (18:1)-PC, palmitoyl (16:0)-oleoyl (18:1)-PC, and myristoyl (14:0)-oleoyl (18:1)-PC as constituents. Thus, as an example, the provided compositions can include palmitoyl 16:0.

ii. Nanoparticles

The term “nanoparticle” refers to a nanoscale particle with a size that is measured in nanometers, for example, a nanoscopic particle that has at least one dimension of less than about 100 nm. Examples of nanoparticles include paramagnetic nanoparticles, superparamagnetic nanoparticles, metal nanoparticles, fullerene-like materials, inorganic nanotubes, dendrimers (such as with covalently attached metal chelates), nanofibers, nanohoms, nano-onions, nanorods, nanoropes and quantum dots. A nanoparticle can produce a detectable signal, for example, through absorption and/or emission of photons (including radio frequency and visible photons) and plasmon resonance.

The nanoparticles can carry the Fab fragments. In some instances, the nanoparticle can be coated with a targeting agent that targets cancer cells.

### E. Phage and Phage Display Libraries

1. Bacteriophage

Disclosed herein are bacteriophage, or phage, that contain a nucleic acid encoding any of the disclosed antibodies, or fragments thereof. For example, the bacteriophage can encode an Fab fragment that binds tRNAᵢ^{Met}, the P4-P6 domain of Tetrahymena Group I intron, the glycine riboswitch from Vibrio Cholerae, or the glycine riboswitch from Fusobacterium Nucleatum.

The disclosed phage and phage libraries are described as encoding different Fab fragments. However, the phage and phage libraries can also encode full length antibodies as well as other antibody fragments that contain the different disclosed Fab fragments.

Disclosed are bacteriophage that encode an Fab fragment that binds tRNAᵢ^{Met}, wherein the Fab have CDRs on the variable regions of the heavy and light chains of the Fab fragment, wherein the CDRs of the light chain comprise one or more of the amino acid sequences SSRYR (SEQ ID NO:1), YGAYRLSSGVPYR (SEQ ID NO:2), and GSSYPV (SEQ ID NO:3) and the CDRs of the heavy chain comprise one or more of the amino acid sequences NFSGSGI (SEQ ID NO:4), GSGSSRGYTR (SEQ ID NO:5), and SGSGSRYAL (SEQ ID NO:6). In one aspect, a bacteriophage that encodes an Fab fragment that binds tRNAᵢ^{Met} can have an Fab that contains one or more of the disclosed CDR sequences for the heavy and light chains or a combination thereof. For example, the encoded Fab fragment can have one, two, or all three of the disclosed light chain sequences alone or in combination with one, two, or all three of the disclosed heavy chain sequences.

Disclosed are bacteriophage that encode an Fab fragment that binds the P4-P6 domain of Tetrahymena Group I intron, wherein the Fab have CDRs on the variable regions of the heavy and light chains of the Fab fragment, wherein the CDRs of the light chain comprise one or more of the amino acid sequences YGYRS (SEQ ID NO:7), YSASGLYRGVPSR (SEQ ID NO:8), and GYRSPV (SEQ ID NO:9) and the CDRs of the heavy chain comprise one or more of the amino acid sequences NLGSGYI (SEQ ID NO:10), SYRPSSGSTR (SEQ ID NO:11), and SYSSRYSYAM (SEQ ID NO:12). In one aspect, a bacteriophage that encodes an Fab fragment that binds the P4-P6 domain of Tetrahymena Group I intron can have an Fab that contains one or more of the disclosed CDR sequences for the heavy and light chains or a combination thereof. For example, the encoded Fab fragment can have one, two, or all three of the disclosed light chain sequences alone or in combination with one, two, or all three of the disclosed heavy chain sequences.

Disclosed are bacteriophage that encode an Fab fragment that binds the P4-P6 domain of Tetrahymena Group I intron, wherein the Fab have CDRs on the variable regions of the heavy and light chains of the Fab fragment, wherein the CDRs of the light chain comprise one or more of the amino acid sequences SVSSA (SEQ ID NO:13), YSASSLYSGVPSR (SEQ ID NO:14), and SYSSPI (SEQ ID NO:15) and the CDRs of the heavy chain comprise one or more of the amino acid sequences NLYSSSI (SEQ ID NO:16), SRSPRSGGTS (SEQ ID NO:17), and RAAGMSTYGF (SEQ ID NO:18). In one aspect, a bacteriophage that encodes an Fab fragment that binds the P4-P6 domain of Tetrahymena Group I intron can have an Fab that contains one or more of the disclosed CDR sequences for the heavy and light chains or a combination thereof. For example, the encoded Fab fragment can have one, two, or all three of the disclosed light chain sequences alone or in combination with one, two, or all three of the disclosed heavy chain sequences.

Disclosed are bacteriophage that encode an Fab fragment that binds the glycine riboswitch from Vibrio Cholerae, wherein the Fab have CDRs on the variable regions of the heavy and light chains of the Fab fragment, wherein the CDRs of the light chain comprise one or more of the amino acid sequences YSYRS (SEQ ID NO:19), YRASRLYGGVPSR (SEQ ID NO:20), and RSSYPV (SEQ ID NO:21) and the CDRs of the heavy chain comprise one or more of the amino acid sequences NFSGSSI (SEQ ID NO:22), YGSPGYGRTS (SEQ ID NO:23), and SSYGSRSGYAM (SEQ ID NO:24). In one aspect, a bacteriophage that encodes an Fab fragment that binds the glycine riboswitch from Vibrio Cholerae can have an Fab that contains one or more of the disclosed CDR sequences for the heavy and light chains or a combination thereof. For example, the encoded Fab fragment can have one, two, or all three of the disclosed light chain sequences alone or in combination with one, two, or all three of the disclosed heavy chain sequences.

Disclosed are bacteriophage that encode an Fab fragment that binds the glycine riboswitch from Vibrio Cholerae, wherein the Fab have CDRs on the variable regions of the heavy and light chains of the Fab fragment, wherein the CDRs of the light chain comprise one or more of the amino acid sequences SGSSY (SEQ ID NO:25), YRASSLSSGVPSR (SEQ ID NO:26), and YSSRLL (SEQ ID NO:27) and the CDRs of the heavy chain comprise one or more of the amino acid sequences NIYRYGI (SEQ ID NO:28), YGSPGSGRTY (SEQ ID NO:29), and RYRYRYRSGL (SEQ ID NO:30). In one aspect, a bacteriophage that encodes an Fab fragment that binds the glycine riboswitch from Vibrio Cholerae can have an Fab that contains one or more of the disclosed CDR sequences for the heavy and light chains or a combination thereof. For example, the encoded Fab fragment can have one, two, or all three of the disclosed light chain sequences alone or in combination with one, two, or all three of the disclosed heavy chain sequences.

Disclosed are bacteriophage that encode an Fab fragment that binds the glycine riboswitch from Fusobacterium Nucleatum, wherein the Fab have CDRs on the variable regions of the heavy and light chains of the Fab fragment, wherein the CDRs of the light chain comprise one or more of the amino acid sequences YSSRY (SEQ ID NO:31), YGAYGLYRGVPYR (SEQ ID NO:32), and SYSYPI (SEQ ID NO:33) and the CDRs of the heavy chain comprise one or more of the amino acid sequences NLGYRYI (SEQ ID NO:34), SGSSGSGSTY(SEQ ID NO:35), and GSSYSYRYAF (SEQ ID NO:36). In one aspect, a bacteriophage that encodes an Fab fragment that binds the glycine riboswitch from Fusobacterium Nucleatum can have an Fab that contains one or more of the disclosed CDR sequences for the heavy and light chains or a combination thereof. For example, the encoded Fab fragment can have one, two, or all three of the disclosed light chain sequences alone or in combination with one, two, or all three of the disclosed heavy chain sequences.

2. Phage Display Library

Disclosed is a phage display library comprising any of the disclosed bacteriophage. The disclosed phage display library can contain bacteriophage that contain nucleic acids that encode any of the disclosed variable region heavy and light chain amino acid sequences. The bacteriophage that make up the phage display library can encode full length antibodies or antibody fragments. For example, the phage display library can contain phage that encode Fab fragments. For example, the phage display library can include one or more of the bacteriophage that encode an Fab fragment that binds tRNAᵢ^{Met}, the bacteriophage that encode an Fab fragment that binds the P4-P6 domain of Tetrahymena Group I intron, the bacteriophage that encodes an Fab fragment that binds the glycine riboswitch from Vibrio Cholerae, the bacteriophage that encodes an Fab Fragment that bind the glycine riboswitch from Fusobacterium Nucleatum.

Disclosed are phage display libraries comprising phage that express the Fab portion of an antibody, wherein all six CDRs of each Fab are mutated, wherein the mutated CDRs comprise only selectively randomized Tyrosine, Serine, Glycine, and Arginine residues. Thus, the phage display library can contain phage in which the CDR-L1 and CDR-L2 are not fixed.

The disclosed phage display libraries were created specifically to increase the binding ability of the encoded Fab fragments to RNA targets. For example, to decrease non-specific binding to RNAs, the phage present in the phage display library can encode an Fab fragment that contains a CDR, wherein the CDR does not contain consecutive arginines.

### F. Methods of Making Phage Display Library

Disclosed are methods of making the disclosed phage display libraries. The phage display libraries were created specifically to increase the binding ability of the encoded Fab fragments to RNA targets. For example, to decrease non-specific binding to RNAs, the phage present in the phage display library can encode an Fab fragment that contains a CDR, wherein the CDR does not contain consecutive arginines.

The disclosed methods of making phage display libraries can include starting with the previously established YSGX library and then making the amino acid composition different for all CDRs. For example, the YSGX library fixed the CDR-L1 and L2. In CDR-L3, H1, and H2 of the YSGX library, amino acids were randomized as 50% tyrosine and 50% serine, and CDR-H3 were randomized as Tyrosine (20%), Serine (15%), Glycine (15%), and 16 others amino acids (3% each) no Cysteine. The disclosed phage display libraries have expanded the randomization to include CDR-L and L2 as well to mutate all 6 CDRs, (L1, L2, L3, H1, H2, H3) in order to enhance Fab binding to RNA targets.

The phage display libraries can contain phage that encode Fab fragments, wherein the Fab fragments have amino acid compositions different for all CDRs. For instance, the CDRs can be comprised of only selectively randomized Tyrosine, Serine, Glysine, and Arginine residues. In one aspect, the randomization can be performed by using two separate degenerate codons, TMT (M=A or C) to encode an equal portion of Tyr, Ser, and VGT (V=A, C, or G) to encode an equal portion of Ser, Gly, and Arg. These two codons can be applied in an alternating fashion in a consecutive CDR sequence. The oligonucleotide primers can be designed in pairs to apply TMT and VGT codons to both odd and even numbered positions. For example, for positions 28-32 of CDR-L1, primers containing either TMTVGTTMTVGTTMT and VGTTMTVGTTMTVGT can be used in the library construction. This design can eliminate the Fabs with consecutive arginines.

The disclosed methods of making phage display libraries provides phage that have affinity to RNA and minimize non-specific nucleic acid binding by reducing the quantity of positively charged amino acids which would reduce specificity of the antibodies or antibody fragments generated. This design is specific for RNA and not proteins which other existing libraries are designed for.

### G. Methods of Treating

1. Methods of Treating with Disclosed Antibodies and Antibody Fragments

Disclosed are methods of treating diseases, infections or disorders with the disclosed antibodies and antibody fragments, wherein the diseases, infections or disorders have an RNA target. For example, cancer and bacterial infections have RNA targets and therefore can be treated with the disclosed RNA-specific antibodies and antibody fragments.

As used herein, Treatment,” “treat,” or “treating” means a method of inhibiting or reducing the effects of a disease or condition. Treatment can also refer to a method of reducing the disease or condition itself rather than just the symptoms. The treatment can be any reduction from native levels and can be but is not limited to the complete ablation of the disease, condition, or the symptoms of the disease or condition. For example, with respect to cancer treatment, the treatment can be inhibition or reduction of tumor proliferation, tumor formation, tumor initiation, metastasis, and/or cancer survival or maintenance is inhibited or enhancement of cancer cell death. Therefore, in the disclosed methods, “treatment” can refer to a 10%, 20%, 30%, 40%, 50%, 60%, 70%, 80%, 90%, or 100% reduction in the severity of an established disease or the disease progression. For example, a disclosed method for reducing the effects of prostate, breast, or colon cancer is considered to be a treatment if there is a 10% reduction in one or more symptoms of the disease in a subject with the disease when compared to native levels in the same subject or control subjects. Thus, the reduction can be a 10, 20, 30, 40, 50, 60, 70, 80, 90, 100%, or any amount of reduction in between as compared to native or control levels. It is understood and herein contemplated that “treatment” does not necessarily refer to a cure of the disease or condition, but an improvement in the outlook of a disease or condition. Although used separately, it is understood that “treating,” “inhibiting,” or “reducing” a cancer refer to the same activity herein.

In one aspect, disclosed herein are methods of treating cancer comprising administering to a subject the any of the disclosed pharmaceutical composition s. For example, the pharmaceutical compositions can contain any of the disclosed antibodies, such as an antibody, antibody fragment, comprising at least one Fab that specifically binds to tRNAᵢ^{Met}, the P4-P6 domain of Tetrahymena Group I intron, the glycine riboswitch from Vibrio cholerae, or the glycine riboswitch from Fusobacterium nucleatum.

Because levels of tRNAiMet can be overexpressed in certain cancers, such as breast cancer, an antibody specific to tRNAiMet can be used to treat the cancer. As disclosed above the compositions and methods disclosed herein can be used to treat, inhibit, and/or reduce any disease where uncontrolled cellular proliferation occurs such as cancers. A non-limiting list of different types of cancers is as follows: lymphomas (Hodgkins and non-Hodgkins), leukemias, carcinomas, carcinomas of solid tissues, squamous cell carcinomas, adenocarcinomas, sarcomas, gliomas, high grade gliomas, blastomas, neuroblastomas, plasmacytomas, histiocytomas, melanomas, adenomas, hypoxic tumours, myelomas, AIDS-related lymphomas or sarcomas, metastatic cancers, or cancers in general.

A representative but non-limiting list of cancers that the disclosed compositions can be used to treat is the following: lymphoma, B cell lymphoma, T cell lymphoma, mycosis fungoides, Hodgkin's Disease, leukemias, myeloid leukemia, bladder cancer, brain cancer, nervous system cancer, head and neck cancer, squamous cell carcinoma of head and neck, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, gastric cancer, colon cancer, cervical cancer, cervical carcinoma, breast cancer (including but not limited to, for example, basal-like breast cancer and luminal (A and B) breast cancer), and epithelial cancer, bone cancers, renal cancer, bladder cancer, genitourinary cancer, esophageal carcinoma, large bowel cancer, metastatic cancers hematopoietic cancers, sarcomas, Ewing's sarcoma, synovial cancer, soft tissue cancers; and testicular cancer. Thus disclosed herein are methods of treating, inhibiting, and/or reducing wherein the cancer is selected form the group of cancers consisting of lymphoma, B cell lymphoma, T cell lymphoma, mycosis fungoides, Hodgkin's Disease, leukemias, myeloid leukemia, bladder cancer, brain cancer, nervous system cancer, head and neck cancer, squamous cell carcinoma of head and neck, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, gastric cancer, colon cancer, cervical cancer, cervical carcinoma, breast cancer (including but not limited to, for example, basal-like breast cancer and luminal (A and B) breast cancer), and epithelial cancer, bone cancers, renal cancer, bladder cancer, genitourinary cancer, esophageal carcinoma, large bowel cancer, metastatic cancers hematopoietic cancers, sarcomas, Ewing's sarcoma, synovial cancer, soft tissue cancers; and testicular cancer.

Thus, the methods of treating cancer can include administering to a subject an effective amount of a pharmaceutical composition, wherein the pharmaceutical composition contains an antibody or antibody fragments that specifically bind tRNAᵢ^{Met} and has CDRs on the variable regions of the heavy and light chains of the Fab, wherein the CDRs of the light chain comprise one or more of the amino acid sequences SSRYR (SEQ ID NO:1), YGAYRLSSGVPYR (SEQ ID NO:2), and GSSYPV (SEQ ID NO:3). In some instances, the antibody or antibody fragments that specifically bind tRNAᵢ^{Met} and has CDRs on the variable regions of the heavy and light chains of the Fab, can contain one or more of the amino acid sequences NFSGSGI (SEQ ID NO:4), GSGSSRGYTR (SEQ ID NO:5), and SGSGSRYAL (SEQ ID NO:6) on the CDRs of the heavy chain. Furthermore, the antibody or antibody fragment within the pharmaceutical compositions can contain any combination of the disclosed heavy and light chain amino acid sequences.

The disclosed methods of treating cancer can include the administration of a pharmaceutical composition containing an antibody or antibody fragment wherein the antibody or antibody fragment contain an Fab that binds tRNAᵢ^{Met} and can inhibit cell proliferation, block binding of tRNAᵢ^{Met} with elongation factors, or prevent tRNAᵢ^{Met} from initiating polypeptide synthesis.

In one aspect, targeting a bacterial riboswitch can be used as a method of treating bacterial infections. Therefore, disclosed are methods of treating a bacterial infection including administering to a subject an effective amount of a pharmaceutical composition, wherein the pharmaceutical composition contains an antibody or antibody fragments that binds the glycine riboswitch from Vibrio Cholerae, wherein the Fab have CDRs on the variable regions of the heavy and light chains of the Fab fragment, wherein the CDRs of the light chain comprise one or more of the amino acid sequences YSYRS (SEQ ID NO:19), YRASRLYGGVPSR (SEQ ID NO:20), and RSSYPV (SEQ ID NO:21)

In some instances, the antibody or antibody fragments that specifically bind the glycine riboswitch from Vibrio Cholerae and has CDRs on the variable regions of the heavy and light chains of the Fab, can contain one or more of the amino acid sequences NFSGSSI (SEQ ID NO:22), YGSPGYGRTS (SEQ ID NO:23), and SSYGSRSGYAM (SEQ ID NO:24) on the CDRs of the heavy chain. Furthermore, the antibody or antibody fragment within the pharmaceutical compositions can contain any combination of the disclosed heavy and light chain amino acid sequences.

Other methods of treating a bacterial infection can include administering to a subject an effective amount of a pharmaceutical composition, wherein the pharmaceutical composition contains an antibody or antibody fragments that binds the glycine riboswitch from Vibrio Cholerae, wherein the Fab have CDRs on the variable regions of the heavy and light chains of the Fab fragment, wherein the CDRs of the light chain comprise one or more of the amino acid sequences SGSSY (SEQ ID NO:25), YRASSLSSGVPSR (SEQ ID NO:26), and YSSRLL (SEQ ID NO:27). In some instances, the antibody or antibody fragments that specifically bind the glycine riboswitch from Vibrio Cholerae and has CDRs on the variable regions of the heavy and light chains of the Fab, can contain one or more of the amino acid sequences NIYRYGI (SEQ ID NO:28), YGSPGSGRTY (SEQ ID NO:29), and RYRYRYRSGL (SEQ ID NO:30) on the CDRs of the heavy chain. Furthermore, the antibody or antibody fragment within the pharmaceutical compositions can contain any combination of the disclosed heavy and light chain amino acid sequences.

Another example of treating bacterial infections by targeting the riboswitch is the method of treating a bacterial infection including administering to a subject an effective amount of a pharmaceutical composition, wherein the pharmaceutical composition contains an antibody or antibody fragments that binds the glycine riboswitch from Fusobacterium Nucleatum, wherein the Fab have CDRs on the variable regions of the heavy and light chains of the Fab fragment, wherein the CDRs of the light chain comprise one or more of the amino acid sequences YSSRY (SEQ ID NO:31), YGAYGLYRGVPYR (SEQ ID NO:32), and SYSYPI (SEQ ID NO:33). In some instances, the antibody or antibody fragments that specifically bind the glycine riboswitch from Fusobacterium Nucleatum and has CDRs on the variable regions of the heavy and light chains of the Fab, can contain one or more of the amino acid sequences NLGYRYI (SEQ ID NO:34), SGSSGSGSTY(SEQ ID NO:35), and GSSYSYRYAF (SEQ ID NO:36) on the CDRs of the heavy chain. Furthermore, the antibody or antibody fragment within the pharmaceutical compositions can contain any combination of the disclosed heavy and light chain amino acid sequences.

2. Methods of Treating with Disclosed Fab Complexes

Disclosed are methods of treating diseases, infections or disorders with the disclosed Fab complexes, wherein the diseases, infections or disorders have an RNA target and the Fab complex contains an Fab specific for the RNA target. For example, cancer and bacterial infections have RNA targets and therefore can be treated with the disclosed RNA-specific Fab complexes. The disclosed methods can treat a wide range of cancers, including but not limited to breast cancer and pancreatic cancer.

Disclosed are methods of treating cancer comprising administering to a subject a pharmaceutical composition containing an Fab complexes containing at least one Fab that specifically binds to tRNAᵢ^{Met}.

Because levels of tRNAᵢ^{Met} can be overexpressed in certain cancers, such as breast cancer, a bi-specific Fab complex containing a therapeutic arm and a delivery arm, wherein the therapeutic arm contains an Fab specific to tRNAᵢ^{Met} can be used to treat the cancer. In one aspect, the delivery arm of the Fab complex can contain an anti-HER2 Fab. The anti-HER Fab allows for delivery of the Fab complex to HER2 positive cancer cells and thus delivers the therapeutic, the tRNAᵢ^{Met} Fab, to the cancer cell. The delivery arm of the Fab complex can contain Fab fragments to other cancer surface receptors, such as but not limited to VEGF, CD30, CD52, CD25, CD3 receptor, CD20, and ErbB2. Other cancer surface receptors are well known in the art.

The tRNAᵢ^{Met} Fab therapeutic arm of the Fab complex can specifically bind tRNAᵢ^{Met} and has CDRs on the variable regions of the heavy and light chains of the Fab, wherein the CDRs of the light chain comprise one or more of the amino acid sequences SSRYR (SEQ ID NO:1), YGAYRLSSGVPYR (SEQ ID NO:2), and GSSYPV (SEQ ID NO:3). In some instances, tRNAᵢ^{Met} Fab therapeutic arm has CDRs on the variable regions of the heavy and light chains of the Fab, wherein the CDRs of the heavy chain contain one or more of the amino acid sequences NFSGSGI (SEQ ID NO:4), GSGSSRGYTR (SEQ ID NO:5), and SGSGSRYAL (SEQ ID NO:6). Furthermore, the tRNAᵢ^{Met} Fab therapeutic arm of the Fab complex within the pharmaceutical compositions can contain any combination of the disclosed heavy and light chain amino acid sequences.

The disclosed methods of treating cancer can include the administration of a pharmaceutical composition containing a Fab complex, wherein the Fab complex has a therapeutic arm and a delivery arm, wherein the therapeutic arm is an Fab that binds tRNAᵢ^{Met} and the subject overexpresses tRNAᵢ^{Met}. The tRNAᵢ^{Met} Fab therapeutic arm, or anti-tRNAᵢ^{Met} Fab, can inhibit cell proliferation, block binding of tRNAᵢ^{Met} with elongation factors, or prevent tRNAᵢ^{Met} from initiating polypeptide synthesis.

### H. Methods of Detecting

Disclosed are methods of detecting RNA using the disclosed RNA-specific antibodies and antibody fragments.

Disclosed are methods of detecting an RNA structure by administering at least one of the disclosed RNA-specific antibodies or antibody fragments, wherein the antibody or antibody fragment is conjugated to a detection moiety. Thus, the methods of detecting include administering the disclosed conjugates, wherein the conjugate contains an RNA-specific antibody or antibody fragment linked or coupled to a detection moiety.

The detection of different RNA structures can be used in the process of crystallization of RNA.

The detection of RNA structures can also be used to study different RNA processes. For example, anti-tRNAᵢ^{Met} antibodies or Fab can be used to study the mammalian translation process.

### I. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits for making Fab complexes, the kit comprising a tRNAᵢ^{Met} Fab and a delivery arm Fab fragment such as anti-HER2 Fab. The kits also can contain linkers and reagents for coupling the two Fab fragments together.

The disclosed kits can also include a tRNAᵢ^{Met} Fab and a detection moiety.

### REFERENCES

1. Zhang, B., Pan, X., Cobb, G. P. & Anderson, T. A. microRNAs as oncogenes and tumor suppressors. Dev Biol 302, 1-12 (2007).
2. Wang, D. et al. Human microRNA oncogenes and tumor suppressors show significantly different biological patterns: from functions to targets. PLoS One 5 (2010).
3. Tsai, M. C., Spitale, R. C. & Chang, H. Y. Long intergenic noncoding RNAs: new links in cancer progression. Cancer Res 71, 3-7 (2011).
4. Huarte, M. & Rinn, J. L. Large non-coding RNAs: missing links in cancer? Hum Mol Genet. 19, R152-61 (2010).
5. Marshall, L., Kenneth, N. S. & White, R. J. Elevated tRNA(iMet) synthesis can drive cell proliferation and oncogenic transformation. Cell 133, 78-89 (2008).
6. Ye, J. D. et al. Synthetic antibodies for specific recognition and crystallization of structured RNA. Proc Natl Acad Sci USA 105, 82-7 (2008).
7. Koldobskaya, Y. et al. A portable RNA sequence whose recognition by a synthetic antibody facilitates structural determination. Nat Struct Mol Biol 18, 100-6 (2011).
8. Ravindran, P. P., Heroux, A. & Ye, J. D. Improvement of the crystallizability and expression of an RNA crystallization chaperone. J Biochem 150, 535-43 (2011).
9. Rould, M. A., Perona, J. J., Soll, D. & Steitz, T. A. Structure of E. coli glutaminyl-tRNA synthetase complexed with tRNA(Gln) and ATP at 2.8 A resolution. Science 246, 1135-42 (1989).
10. Lu, M. & Steitz, T. A. Structure of Escherichia coli ribosomal protein L25 complexed with a 5S rRNA fragment at 1.8-A resolution. Proc Natl Acad Sci USA 97, 2023-8 (2000).
11. Kirkwood, J. M. et al. Immunotherapy of cancer in 2012. CA Cancer J Clin (2012).
12. Hayashita, Y. et al. A polycistronic microRNA cluster, miR-17-92, is overexpressed in human lung cancers and enhances cell proliferation. Cancer Res 65, 9628-32 (2005).
13. Gutschner, T., Bass, M. & Diederichs, S, Noncoding RNA gene silencing through genomic integration of RNA destabilizing elements using zinc finger nucleases. Genome Res 21, 1944-54 (2011).
14. Fellouse, F. A., Wiesmann, C. & Sidhu, S. S. Synthetic antibodies from a four-amino-acid code: a dominant role for tyrosine in antigen recognition. Proc Natl Acad Sci USA 101, 12467-72 (2004).
15. Fellouse, F. A. et al. Molecular recognition by a binary code. J Mol Biol 348, 1153-62 (2005).
16. Sidhu, S. S. et al. Phage-displayed antibody libraries of synthetic heavy chain complementarity determining regions. J Mol Biol 338, 299-310 (2004).
17. Sidhu, S. S., Lowman, H. B., Cunningham, B. C. & Wells, J. A. Phage display for selection of novel binding peptides. Methods Enzymol 328, 333-63 (2000).
18. Agris, P. F. et al. Experimental models of protein-RNA interaction: isolation and analyses of tRNA(Phe) and U1 snRNA-binding peptides from bacteriophage display libraries. J Protein Chem 18, 425-35 (1999).
19. Mian, I. S., Bradwell, A. R. & Olson, A. J. Structure, function and properties of antibody binding sites. J Mol Biol 217, 133-51 (1991).
20. Braunagel, M. & Little, M. Construction of a semisynthetic antibody library using trinucleotide oligos. Nucleic Acids Res 25, 4690-1 (1997).
21. Cabarcas, S. & Schramm, L. RNA polymerase III transcription in cancer: the BRF2 connection. Mol Cancer 10, 47 (2011).
22. Winter, A. G. et al. RNA polymerase III transcription factor TFIIIC2 is overexpressed in ovarian tumors. Proc Natl Acad Sci USA 97, 12619-24 (2000).
23. Pavon-Eternod, M. et al. tRNA over-expression in breast cancer and functional consequences. Nucleic Acids Res 37, 7268-80 (2009).
24. Zetterberg, A. & Killander, D. Quantitative cytophotometric and autoradiographic studies on the rate of protein synthesis during interphase in mouse fibroblasts in vitro. Exp Cell Res 40, 1-11 (1965).
25. Francis, M. A. & Rajbhandary, U. L. Expression and function of a human initiator tRNA gene in the yeast Saccharomyces cerevisiae. Mol Cell Biol 10, 4486-94 (1990).
26. Tomlinson, V. A. et al. Translation elongation factor eEF1A2 is a potential oncoprotein that is overexpressed in two-thirds of breast tumours. BMC Cancer 5, 113 (2005).
27. Frye, M. & Watt, F. M. The RNA methyltransferase Misu (NSun2) mediates Myc-induced proliferation and is upregulated in tumors. Curr Biol 16, 971-81 (2006).
28. Shin, S. H. et al. Implication of leucyl-tRNA synthetase 1 (LARS1) over-expression in growth and migration of lung cancer cells detected by siRNA targeted knock-down analysis. Exp Mol Med 40, 229-36 (2008).
29. Wong, I. & Lohman, T. M. A double-filter method for nitrocellulose-filter binding: application to protein-nucleic acid interactions. Proc Natl Acad Sci USA 90, 5428-32 (1993).
30. Cochrane, J. C., Batey, R. T. & Strobel, S. A. Quantitation of free energy profiles in RNA-ligand interactions by nucleotide analog interference mapping. Rna 9, 1282-9 (2003).
31. Sherman, E. M., Esquiaqui, J., Elsayed, G. & Ye, J. D. An energetically beneficial leader-linker interaction abolishes ligand-binding cooperativity in glycine riboswitches. Rna 18, 496-507 (2012).
32. Guillon, L., Schmitt, E., Blanquet, S. & Mechulam, Y. Initiator tRNA binding by e/aIF5B, the eukaryotic/archaeal homologue of bacterial initiation factor IF2. Biochemistry 44, 15594-601 (2005).
33. Hamrita, B. et al. An elongation factor-like protein (EF-Tu) elicits a humoral response in infiltrating ductal breast carcinomas: An immunoproteomics investigation. Clin Biochem 44, 1097-104 (2011).
34. Cassinelli, G. et al. Inhibition of c-Met and prevention of spontaneous metastatic spreading by the 2-indolinone RPI-1. Mol Cancer Ther 5, 2388-97 (2006).
35. Morris, M. C., Depollier, J., Mery, J., Heitz, F. & Divita, G. A peptide carrier for the delivery of biologically active proteins into mammalian cells. Nat Biotechnol 19, 1173-6 (2001).
36. Schwarze, S. R. & Dowdy, S. F. In vivo protein transduction: intracellular delivery of biologically active proteins, compounds and DNA. Trends Pharmacol Sci 21, 45-8 (2000).
37. Rizk, S. S. et al. An engineered substance P variant for receptor-mediated delivery of synthetic antibodies into tumor cells. Proc Natl Acad Sci USA 106, 11011-5 (2009).
38. Bale, S. S. et al. Nanoparticle-mediated cytoplasmic delivery of proteins to target cellular machinery. ACS Nano 4, 1493-500 (2010).
39. Iyer, A. K. et al. Novel human single chain antibody fragments that are rapidly internalizing effectively target epithelioid and sarcomatoid mesotheliomas. Cancer Res 71, 2428-32 (2011).
40. Holmes, D. Buy buy bispecific antibodies. Nat Rev Drug Discov 10, 798-800 (2011).
41. Thakur, A. & Lum, L. G. Cancer therapy with bispecific antibodies: Clinical experience. Curr Opin Mol Ther 12, 340-9 (2010).
42. Chames, P. & Baty, D. Bispecific antibodies for cancer therapy: the light at the end of the tunnel? MAbs 1, 539-47 (2009).
43. Loo, L., Robinson, M. K. & Adams, G. P. Antibody engineering principles and applications. Cancer J 14, 149-53 (2008).
44. Jung, G., Honsik, C. J., Reisfeld, R. A. & Muller-Eberhard, H. J. Activation of human peripheral blood mononuclear cells by anti-T3: killing of tumor target cells coated with anti-target-anti-T3 conjugates. Proc Natl Acad Sci USA 83, 4479-83 (1986).
45. Eigenbrot, C., Randal, M., Presta, L., Carter, P. & Kossiakoff, A. A. X-ray structures of the antigen-binding domains from three variants of humanized anti-p185HER2 antibody 4D5 and comparison with molecular modeling. J Mol Biol 229, 969-95 (1993).
46. Wang, Y. et al. Tumor delivery of antisense oligomer using trastuzumab within a streptavidin nanoparticle. Eur J Nucl Med Mol Imaging 36, 1977-86 (2009).
47. Moor, N. A., Ankilova, V. N. & Lavrik, O. I. Recognition of tRNAPhe by phenylalanyl-tRNA synthetase of Thermus thermophilus. Eur J Biochem 234, 897-902 (1995).
48. Peterson, E. T. & Uhlenbeck, O. C. Determination of recognition nucleotides for Escherichia coli phenylalanyl-tRNA synthetase. Biochemistry 31, 10380-9 (1992).
49. Harrington, K. M., Nazarenko, I. A., Dix, D. B., Thompson, R. C. & Uhlenbeck, O. C. In vitro analysis of translational rate and accuracy with an unmodified tRNA. Biochemistry 32, 7617-22 (1993).
50. Curnow, A. W., Kung, F. L., Koch, K. A. & Garcia, G. A. tRNA-guanine transglycosylase from Escherichia coli: gross tRNA structural requirements for recognition. Biochemistry 32, 5239-46 (1993).
51. Rudnick, S. I. et al. Influence of affinity and antigen internalization on the uptake and penetration of Anti-HER2 antibodies in solid tumors. Cancer Res 71, 2250-9 (2011).
52. Rudnick, S. I. & Adams, G. P. Affinity and avidity in antibody-based tumor targeting.

### EXAMPLES

### J. Example 1

### Designing Phage Libraries and Identifying tRNAᵢ^{Met}—Specific Fab

### 1. Background

The ncRNAs (non-coding RNAs) fulfill a wide range of functions, including chromatin organization, transcriptional termination, splicing, RNA editing and processing, translational inhibition, and epigenetic regulation. The importance of ncRNAs in these key cellular processes has rendered them valuable and sometimes irreplaceable drug targets. Numerous lincRNAs (long intergenic ncRNAs) have been shown to lead to tumorigenesis. HOTAIR, for instance, targets polycomb repressive complex 2 (PRC2) genome-wide to alter H3K27 methylation and gene expression patterns, resulting in increased cancer invasiveness and metastasis in breast carcinomas3. Overexpression and amplification of miRNAs that target tumor-suppressor genes can also lead to cancer development. One of the well-studied oncomir clusters, miR-17˜92, has been shown to be overexpressed in human lung cancers. Mediating the biogenesis of its oncomir product miR18a can be achieved by blocking the apical loop of miR18a in the primary polycistron, suggesting primary miRNA as a potential cancer drug target₁₂. Recently, overexpression of tRNAᵢ^{Met} was shown to drive cell proliferation, cause malignant transformation of immortalized mouse embryonic fibroblasts (MEF) and stimulate tumor formation in mices. Most of these ncRNAs are highly structured and interact with proteins to carry out their biological functions, making RNA-protein interfaces superior targets for therapeutic intervention. However, targeting structured RNAs has been sporadic and there lacks a systematic way of producing RNA-binding agents to mediate RNA-protein interfaces. Application of targeting structured RNAs with antibodies can be used to develop variety of biochemical tools, such as RNA crystallization chaperones-s, RNA immunoprecipitation, and RNA cellular imaging.

### 2. Fab-RNA Recognition and Phage Libraries Tailored for Anti-RNA Fab Selection

As there is a lack of a robust method of targeting structured ncRNAs, ncRNAs can be targeted with antibody Fabs for future drug development. Phage display techniques can be used to select anti-RNA Fabs from a synthetic naïve library. Synthetic Fab libraries are generally better than libraries from natural immune systems because they are unbiased toward any given antigen structure, allowing successful selection for diversified targets. In addition, synthetic Fab libraries use a defined Fab scaffold, allowing improvement of the library through CDR reengineering as well as consistent overexpression of the Fab proteins. CDR design is essential to the success of synthetic Fab libraries in phage display selections. A sophisticated design of the variable sites and amino acid composition in CDR randomization can lead to a general anti-RNA Fab library capable of producing Fab binders for most structured RNAs.

The key to a successful synthetic Fab library is the CDR design. It has been demonstrated that Fab libraries with CDRs randomized by a preselected “reduced genetic code” instead of all 20 amino acids produce high-affinity and high-specificity Fabs against protein antigens(14-16). This reduced genetic code design allows expansion of the randomized sites in phage display libraries without significantly increasing the theoretical library size, leading to functionally enriched libraries. The YSGX library and the YSGRX library have been applied to anti-RNA Fab selection with reasonable success(6,7). However, these libraries were not designed for RNA antigens, careful design needs to be employed to improve the efficacy of Fab libraries toward RNA antigens.

i. Construction of the YSGR Min Library for Optimization of Variable Sites.

Although not randomized in the YSGX or YSGRX libraries, light chain CDR L and L2 have been shown to play an important role in Fab-RNA interfaces(6,7). The crystal structures of Fab2/P4-P6 and BL3-6/Class I ligase were examined and randomized the solvent exposed and potential contact residues in CDR-L1 and L2 in addition to the variable sites chosen for the YSGRX library(6,7). This added 13 additional variable sites to the library. To reduce the gap between theoretical size and practical size (typically ˜10¹⁰) of the library, the variable sites were randomized with the tetranomial code: YSGR. The diversity was incorporated into the Fab genes through oligonucleotides primers via Kunkel mutagenesis as described (17). For synthetic ease and economical reasons, the degenerate codon method was used to establish sequence diversity in the primer design. To exclude cysteine or stop codon from the design, two separate degenerate codons, [TMT](brackets indicate degenerate codons) to encode YS, and [VGT] to encode SGR were used. These two codons were applied in an alternating manner in a consecutive CDR sequence and the oligonucleotide primers were designed in pairs to apply the degenerate codons to both odd and even numbered positions. For example, for position 91-94 of CDR-L3, both [TMT][VGT][TMT][VGT] and [VGT][TMT][VGT][TMT] will be used in the library construction. This has a serendipitous advantage. As the YSGRX library often produces Fabs with consecutive arginines in the CDR-H3 and may increase non-specific binding to RNAs, this design can eliminate the Fabs with consecutive arginines by design. The YSGR Min library has been constructed with excellent diversity (3.2×10⁹) and selected against three well-defined RNA targets, the P4-P6 domain, VCIII, and FNIII glycine riboswitches. Filter binding assay(6) of the expressed Fabs showed that the YSGR Min library has yielded specific Fabs for all three RNA targets with nanomolar affinities (Table 1). The quantitative specificity values measured by filter binding assays in the presence of competitive RNAs showed these Fabs are slightly more specific than Fabs selected from the YSGRX library, possibly due to avoiding consecutive arginines.

TABLE 1
Affinities and amino acid sequences of specific RNA binding Fabs from
the YSGR Min library.

| | | K_{d} | | | | | | |
|---|---|---|---|---|---|---|---|---|
| Target | clone | (nM) | L1 | L2 | L3 | H1 | H2 | H3 |
| P4-P6 | 8 | 53 ± 11 | YGYRS | YSASGLYRGVPSR | GYRSPV | NLGSGYI | SYRPSSGSTR | SYSSRYSYAM |
| | 11 | 20 ± 7 | SVSSA | YSASSLYSGVPSR | SYSSPI | NLYSSSI | SRSPRSGGTS | RAAGMSTYGF |
| VCIII | 29 | 38 ± 9 | YSYRS | YRASRLYGGVPSR | RSSYPV | NFSGSSI | YGSPGYGRTS | SSYGSRSGYAM |
| | 30 | 22 ± 5 | SGSSY | YRASSLSSGVPSR | YSSRLL | NIYRYGI | YGSPGSGRTY | RYRYRYRSGL |
| FNIII | 16 | 160 ± 35 | YSSRY | YGAYGLYRGVPYR | SYSYPI | NLGYRYI | SGSSGSGSTY | GSSYSYRYAF |

ii. Characterization of the YSGR Min Library to Optimize Key Variable Sites and Define Basal Amino Acid Codes.

The fact that the YSGR min library has yielded specific Fabs for all three RNA targets indicates that the YSGR tetranomial code is sufficient for RNA binding with medium to high affinity and specificity, demonstrating YSGR code as a good basal “reduced genetic code” for anti-RNA Fab libraries.

iii. Expanded Minimal Code Libraries Designed to Optimize the “Reduced Genetic Code.”

Additional amino acids can be added to the library design and evaluated for their effect in RNA binding affinity and specificity. Agris and coworkers(18) selected peptides of 15 amino acids binding to the ˜160 nt U1 snRNA. Out of the enormous list of isolated sequences, the frequency of amino acids on the U1-snRNA-binding peptides was summarized as the following: S₁₁₆W₉₇G₉₄F₈₆V₈₃L₈₃A₇₃P₆₂Y₆₁T₅₄H₄₃R₄₀M₁₂N₁₁I₈E₈C₇Q₃D₃K₀. Trp ranks second in the frequency map which is similar to the result of Trp as a frequent contact residue in the Kabat data base(19). Trp can be added to the minimal code library by replacing [VGT] with [DGK], which encodes for SGRWC. Furthermore, the three next frequent amino acids (F, V, and L), can be replaced [VGT] with [DKK], which encodes for SGRWFVLIMC. Library construction and selection can be carried out as described above. The frequency of the four amino acids WFVL in the selected sequences can be analyzed and mutational studies can be carried out to define their roles in Fab-RNA binding.

iv. Trinucleotide Code Library, the Ultimate Anti-RNA Fab Library.

With the study of the above minimal and expanded minimal code libraries, optimized variable sites and “reduced genetic code” are obtained. This information can be applied to design an optimal Fab library specifically for RNA targets. Less important variable sites will be randomized with only YS binomial codes and the more important ones will be randomized with the optimal “reduced genetic code.” It may be very challenging to include the optimal reduced genetic code with simple degenerate codons. Therefore, the trinucleotide method can be used to synthesize the oligonucleotides needed for library construction as it allows for accurate control of the type and frequency of the amino acid composition(20). This library can serve as the ultimate library for anti-RNA Fab selection and can faithfully produce high-affinity and high specificity RNA binding Fabs without affinity maturation.

The YSGR Min, expanded minimal code, and the ultimate anti-RNA Fab libraries can be obtained. These libraries can be used in phage displayed Fab selection against oncogenic RNAs as well as other structured RNAs. In addition, they can provide detailed information of the Fab-RNA interfaces to study the protein-RNA interaction in general.

The YSGR Min library can provide medium to high-affinity Fabs for a diverse set of RNAs. Anti-RNA Fab libraries can produce high-affinity and high-specificity RNA binding Fabs without affinity maturation to almost all structured RNAs. A small percentage (a total of 30%˜50%) of the rest of the amino acids (20 amino acids minus expanded minimal code and Cys) can be added to the randomization repertoire for the more important variable sites if need be. This can be achieved readily with the trinucleotide method, and can improve the library efficacy for general RNA targets.

### 3. Specific Fabs Binding to Initiator tRNA

Overexpression of RNA polymerase III transcripts has been observed in a wide range of tumors including breast, cervix, esophagus, lung, ovary, parotid, and tongue(21). One type of its transcripts, tRNAs were shown to be overexpressed consistently in human ovarian cancers by RT-PCR analysis(22). In breast tumors versus normal breast tissues, both nuclear- and mitochondrial-encoded tRNAs were shown to be up to 10-fold higher(23). Furthermore, over-expression of tRNAs is not just a by-product of the carcinogenic transformation: modest overexpression of tRNAᵢ^{Met} was shown to drive cell proliferation, cause malignant transformation of immortalized MEF cells and stimulate tumor formation in mice(5). These results indicate that tRNAᵢ^{Met} can be a potential therapeutic target for breast cancer. Discussed herein are methods of targeting tRNAᵢ^{Met} with synthetic antibodies. Specific Fabs binding tRNAᵢ^{Met} can be produced and can mediate tRNAᵢ^{Met}-involved biological processes related to cancer.

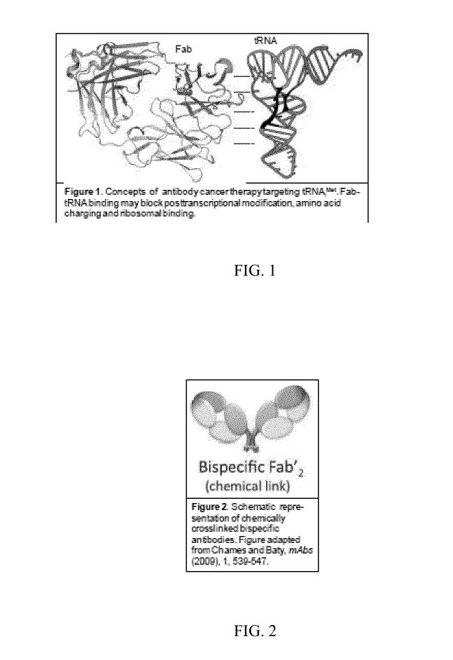

Using immortalized MEF cells stably transfected with tRNAᵢ^{Met} gene, White and colleagues showed that modest overexpression of tRNAᵢ^{Met} can significantly increase protein synthesis (for c-Myc and Cyclin D1), drive cell proliferation and cause focus formation while MEF cells transfected with empty vector or elongator tRNA (tRNAₑ^{Met}) failed to do sos. Furthermore, injection of tRNA_{iMet}-transfected MEF cells caused mice to form tumors between weeks 6-12 while the control mice with empty vector or tRNAₑ^{Met} showed no signs of tumor even after seven months. This indicates that tRNAᵢ^{Met} can be a potential candidate for a diagnostic marker and therapeutic target. The oncogenic role of tRNAᵢ^{Met} is directly correlated to protein synthesis. As protein comprises 80%-90% of a cell's dry mass, translation rate plays a dominant role in cell growth and proliferation₂₄. The rate determining step of translation is translation initiation, which depends on the availability of tRNAᵢ^{Met} and its binding to the small ribosomal subunit25. The importance of translation hyperactivity in cell proliferation and transformation is supported by the oncogenicity of eEF1A, which recruits amino-acylated tRNAs to the ribosome in both translation initiation and elongation26. The oncogenicity of tRNA methyltransferase Misu in breast and colon cancers(27) and tRNA synthetase LARS1 in lung cancer₍₂₈₎ indicates that aberrant tRNA maturation can induce malignant transformation as well. These observations indicate the validity of targeting tRNAᵢ^{Met} for cancer therapy as it can have multiple benefits in blocking posttranscriptional modification, amino acid charging, and ribosomal binding (FIG. 1).

The human tRNAᵢ^{Met} gene is constructed with PCR methods from DNA oligonucleotides. The DNA genes are cloned into pUC19 vector and amplified by JM109 cells. tRNAᵢ^{Met} transcript is prepared by in vitro transcription and purified by dPAGE. For selection, a 29 nt tag was engineered at the 3′ of the RNA, which allowed immobilization on the solid support after complexing with complementary biotinylated DNA oligonucleotides. Phage displayed Fab selection was carried out using published protocols(6). Selection using the YSGRX library and YSGR Min library is performed. With 50 nM tRNAᵢ^{Met} target and 50 μg/ml sheared salmon DNA as a competitor, we have obtained an enrichment (colony number of selection output vs. non-RNA control) of 443 in the fourth round of selection, indicating successful selection. Phage ELISA showed that 42/48 are positive clones. Of the ten clones with best signal to control ratios sent for sequencing, one had a unique sequence (Table 2). Competitive phage ELISA showed this clone binds tRNAᵢ^{Met} with a nanomolar affinity.

TABLE 2
Affinities and amino acid sequences of specific
Fabs binding initiator tRNA^{a}

| Target | clone | K_{d} (nM) | L3 | H1 | H2 | H3 |
|---|---|---|---|---|---|---|
| tRNAᵢ^{Met} | 1 | 53 ± 11 | SSYSSS | SISSSSI | YIYPSSGSTY | YRYRSGHAL |

^{a}CDR-L1 and L2 are not shown as they are not randomized.

i. Selection, Expression, and Characterization of tRNAiMet-Binding Fabs.

Anti-tRNAᵢ^{Met} Fabs can be selected using YSGR Min and future libraries. Phage ELISA can be used to identify positive clones. Unique (identified by sequencing) and positive clones can be expressed as soluble Fabs using the modified protocol for Fab expression(8). Filter binding assays can be carried out with ³²P labeled tRNAᵢ^{Met} according to standard protocol to obtain Fab-RNA binding affinities(6,29,30). Briefly, ³²P labeled RNA can be incubated with various amount of Fab and passed through nitrocellulose and Hybond N+ membrane sequentially under vacuum. Quantification of Fab bound and free RNA captured on the nitrocellulose and Hybond N+ membrane respectively under different Fab concentrations can provide binding affinities. Specificity of the Fabs can be measured when binding these Fabs to non-cognate RNA targets. Fab RNA complexation can be visualized with native gel electrophoresis using published protocols(8).

ii. Investigating the Function of Anti-tRNA Fabs in Cell-Free Assays.

A solution based structure probing approach, the hydroxyl radical footprinting assay(6,31), can be used to reveal the binding site of Fab on the tRNA, which helps predict the possible tRNA metabolic pathways that anti-tRNA Fabs may interfere with. Specific Fabs can be further evaluated for their ability to compete for tRNA (or aminoacyl-tRNA) binding with tRNA metabolic proteins such as tRNA methyltransferase(27), tRNA synthetase(28), eEF1A(26), eIF2(32), eIF5B(32), EF-Tu(33) etc. This can be achieved through immunoprecipitation of the tRNA metabolic proteins in the presence and absence of anti-RNA Fabs and analyzing the complex by antibodies against these tRNA metabolic proteins using western blot assay. Specific Fabs can also be tested for their ability to reduce the efficiency of human in vitro translation system. Using 1-step human in vitro protein expression kit (Thermo Scientific), whether GFP reporter expression can be reduced by the addition of our tRNAᵢ^{Met}-binding Fabs in a concentration dependent manner can be examined.

iii. Investigating the Function of Anti-tRNA Fabs in Ex Vivo Assays.

Therapeutic antibodies have been delivered into cancer cells with various efficiencies and specificities via conjugation or complexation approaches(34-39). To further aid in the delivery efficiency and specificity of antibody fragments into cancer cells and tumors, the anti-tRNAᵢ^{Met} Fabs can be engineered into bispecific antibodies. Holding great promise, bispecific antibodies(40,41) serve two critical functions: one Fab (therapeutic arm) to recognize the oncogenic RNA to achieve therapeutic function and the other Fab (delivery arm) to recognize the cancer cell surface receptor to achieve specific and efficient cancer cell delivery. Multiple forms of bispecific antibodies have been created by research labs and pharmaceutical companies(42,43). The bispecific Fab′2 antibodies (FIG. 2) can be generated using chemical crosslinking as described(44). Readily available through CDR swapping on our Fab scaffold(45), anti-HER2 Fab 4D5 can be used as the delivery arm since the HER2-binding antibody has been used successfully in delivering antisense drugs into breast tumor in a mouse model(46). The bispecific Fab′2 antibodies with both anti-tRNAiMet and anti-HER2 Fabs can be delivered to HER2+ breast cancer cell line SUM 19046 and test their effect on cancer related c-Myc/Cyclin D1 mRNA expression, protein expression, cell proliferation rate and focus formation(5). Other HER2+ cancer cell lines (such as L3.6pL pancreatic cancer cells) can also be used to test the effect of anti-tRNAᵢ^{Met} antibodies in non-breast cancer cells.

Specific Fabs binding to tRNAᵢ^{Met} can be obtained and some of them can interfere with in vitro translation. These Fabs can be engineered into bispecific antibodies with an anti-HER2 Fab arm and delivered into HER2+ cancer cell lines. Those bispecific complexes that have anti-cancer activities can be used as anti-cancer drugs.

Human tRNAiMet with proper base and sugar modifications can be obtained through overexpression in yeast as reported(25) and used in Fab selection. The stability and fate of the internalized Fabs are addressed by the showing that internalized specific Fab has can bind cognate actin cytoskeleton in tumor cells(37). In addition, the affinity of anti-HER2 Fabs can be altered to achieve optimized internalization and drug efficacy(51,52).

### K. Example 2

### YSGRMin Fab Library and RNA Construct Synthesis for Chaperone-Assisted RNA Crystallography

In the human genome, 90% of genetic information is transcribed but only a fraction of the subsequent RNA is translated into proteins. RNAs which are not translated into proteins are deemed noncoding RNAs. Relatively little is known about the vast array of noncoding RNAs, although these molecules perform a variety of functions. RNA crystallography is used to study RNA tertiary structure, which gives insight to the function of these noncoding RNAs. Complications associated with RNA crystallography arise due to RNA's instability, possible conformational heterogeneity, and lack of surface functional group diversity.

A novel technique, Chaperone-Assisted RNA Crystallography (CARC), can aid in crystallization of RNA; synthetic antigen binding fragments (Fabs) act as crystallization chaperones in complex with certain RNAs. A randomized library of synthetic Fabs enriched in all six Complementarity Determining Regions (CDRs, ligand binding regions) with tyrosine, serine, glycine, and arginine residues was constructed; these residues are chosen to hone this YSGRMin, minimal codon/residue library, for RNA binding. This library was screened via phage display selection against three RNA targets: P4P6 (part of self splicing Group I introns, ribozymes, here used as a standard) and two bacterial species' glycine riboswitch constructs FNIII and VCLD1 (this riboswitch performs 5′ mRNA UTR regulatory feedback). After several steps, four YSGRMin Fabs were isolated as specifically binding to the targets, and their binding affinities, Kds, have been obtained, ˜20-150 nM.

### 1. Materials and Methods

Current antibody technologies target proteins; these antibodies are used for many applications. In contrast, RNA-targeting antibody technologies are lacking due to RNA's low immunogenicity in vivo. In vitro, phage displayed synthetic Fab libraries can be screened to obtain RNA-binding antibodies.

YSGRMin Library Design was based on previous work and amino acids Y, S, G, or R and specific Fab residue positions were chosen. All six CDRs were mutagenized with a total of sixteen primers via Kunkel mutagenesis.

Three RNA targets, P4P6, FNIII, and VCLD1, created in vitro via molecular biology, were chosen to perform phage display selection, isolating Fabs that bind to them. Via ELISA, ˜10 specifically binding Fabs for each target were chosen and sequenced from a random pool taken from the selection output. All nondegenerate Fabs were put through molecular biology as plasmids to render them expressible in E. coli 34B8 cells.

After purification with affinity followed by cation exchange column chromatography, Fabs were complexed with cognate targets and run in a native gel to check binding specificity. Specifically binding Fabs from native gels were assayed via dot blot to obtain exact Kds.

### 2. Results

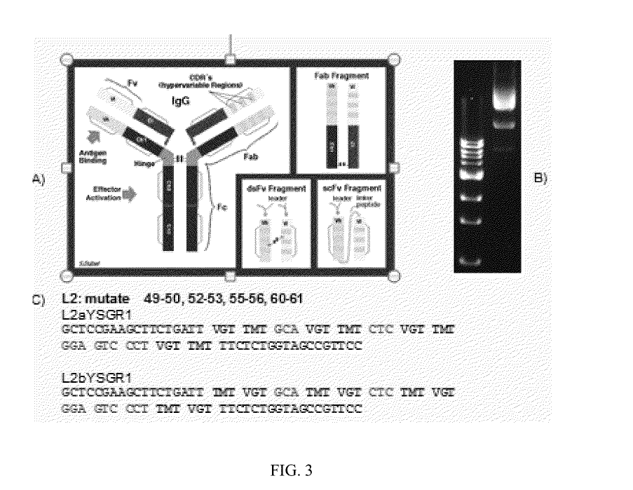

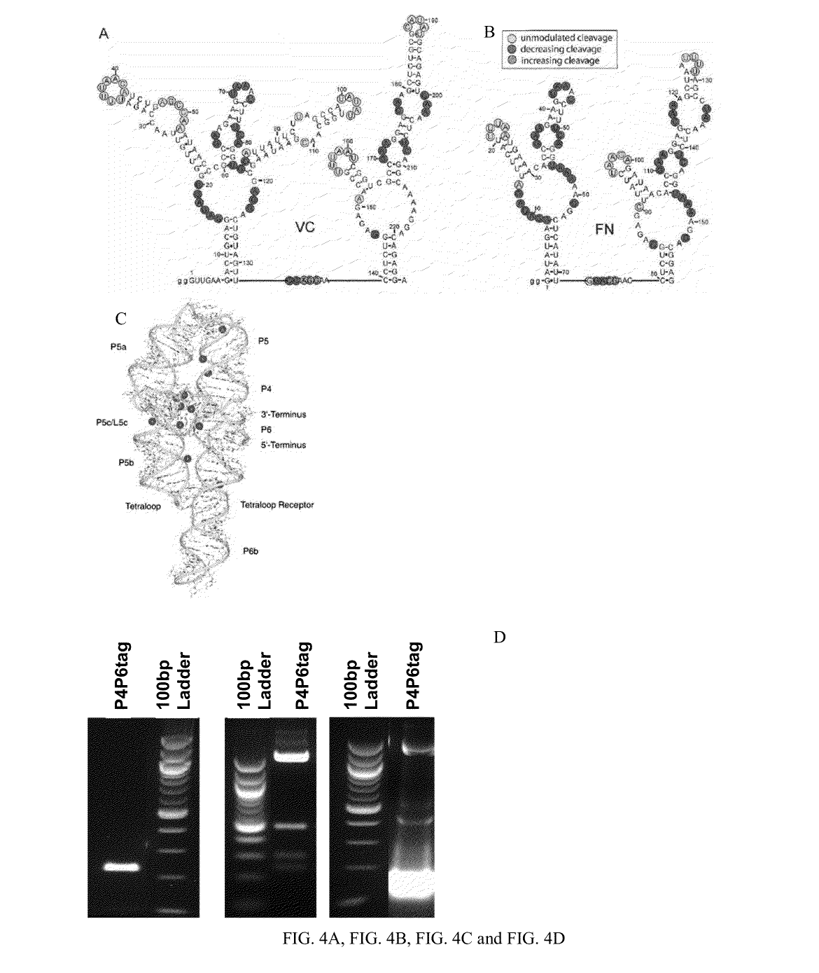

The YSGR Minimal Codon Library of Fab fragments was synthesized, as were its RNA targets (FIG. 3-4).

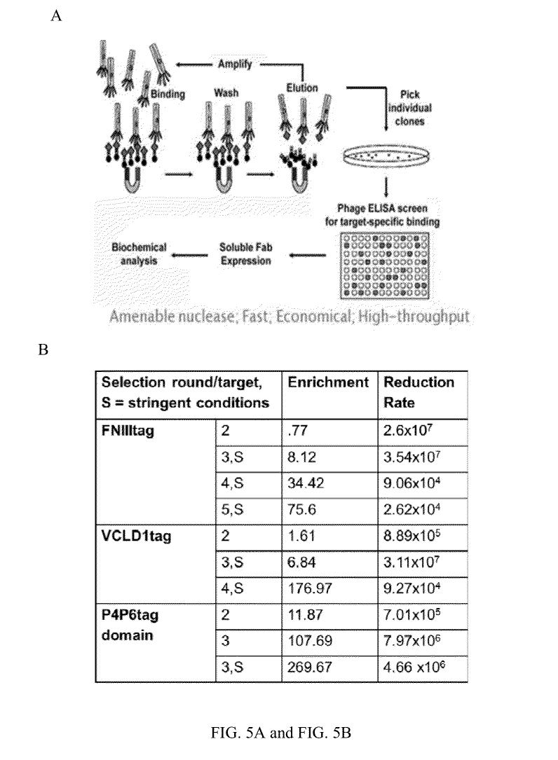

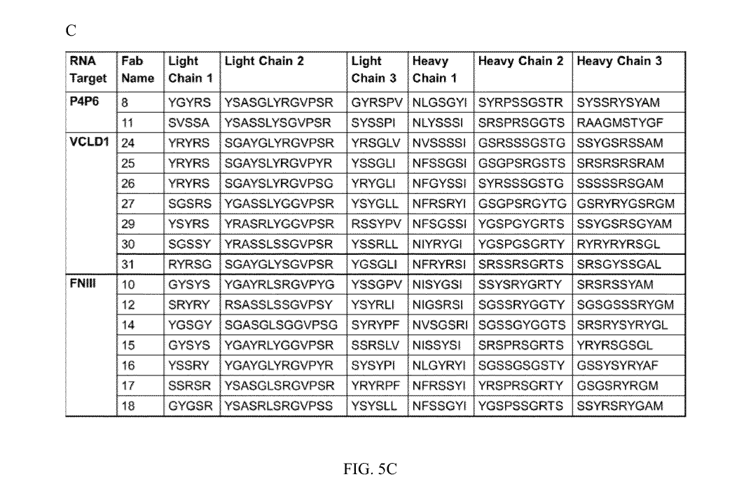

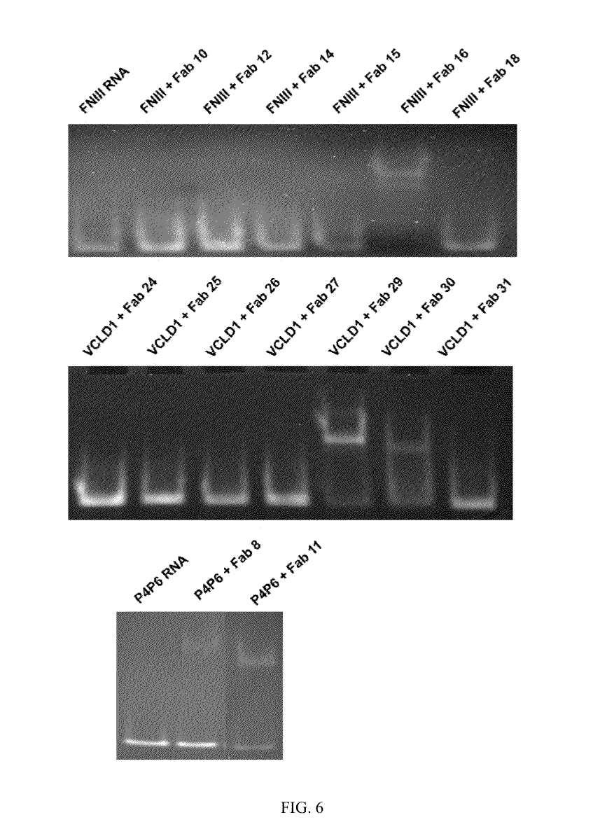

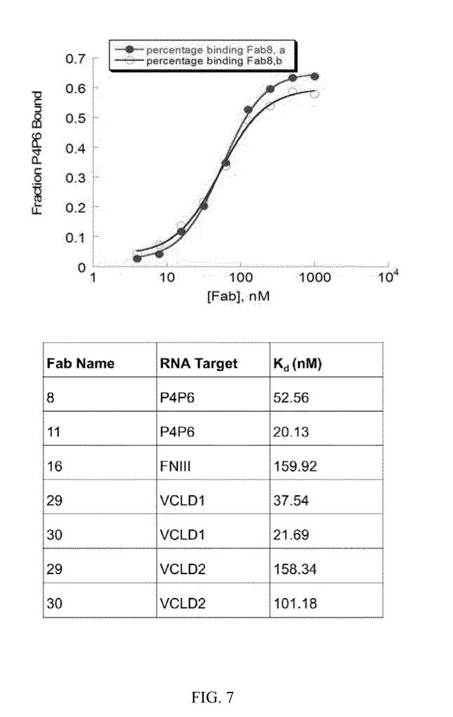

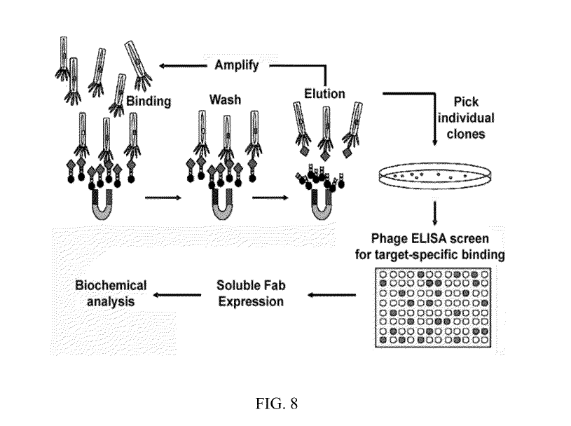

Fabs within the library which bound their target RNA were selected, screened and sequenced (FIG. 5). Unique Fabs were expressed as soluble proteins and purified. Expressed Fabs were assayed for specificity and binding affinity through EMSA and Dot Blot techniques, respectively (FIG. 6-7), displaying acceptable nanomolar Kd values.

A minimal codon design within a Fab library has proven here to be successful for enriching Fabs with RNA-specific residues in CDRs, as several Fabs with reasonable nanomolar Kds were isolated. This minimal codon design paired with the phage display initial screening process can be viewed as a model system for obtaining anti-RNA antibodies which can then be used for a variety of clinical and biomedical applications. Furthermore, RNA, though unable to be immunogenic in a host mammal due to the immune system's generated nucleases, can indeed be an antibody/Fab′ s antigen ligand if handled with care to avoid nucleases and innate chemical lability.

### 3. The Validation of Specificity of a Novel Anti-tRNAiMet Fab Complex for Use in HER2/Neu Positive Human Breast Cancer Immunotherapy

Trastuzumab (Herceptin) is a monoclonal antibody that targets the extracellular and intracellular domains of the HER2/neu receptor, a human epithelial growth factor receptor found on breast cancer cells. Concurrently, the overexpression of initiator tRNA has been implicated as a pro-oncogenic trigger in precancerous cells in mice and correlative in human breast cancer.

Two Fab have been crosslinked together while retaining the binding profile of both Fab globulins individually. The delivery arm, anti-HER2 Fab, is designed for translocation of the bi-specific Fab complex into the cancer cell; while the therapeutic arm, anti-tRNAi Met Fab will bind and sequester the overexpressed tRNAi Met.

An anti-HER2 Fab was synthesized and an elongator tRNA^{Met} was produced for further validation studies. The validation studies include determination of the binding affinity (Kd---) of the anti-tRNAᵢ^{Met} to tRNAᵢ^{Met} versus the elongator tRNA^{Met}.

Elongator tRNA^{Met} and anti-HER2 Fab were produced from C209 P4-P6 Fab.

A novel anti-tRNAi^{Met} binding affinity and specificity against tRNAᵢ^{Met} and elongator tRNA^{Met} was validated. Specific Fab binding to initiator tRNA was demonstrated, as well as using these Fab to mediate biological processes related to cancer.

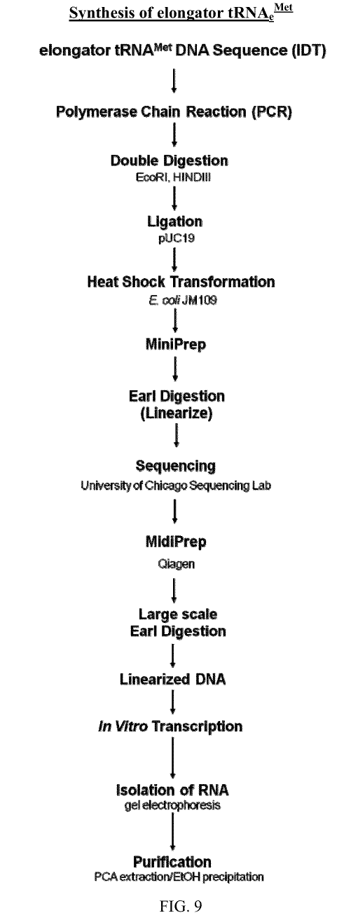

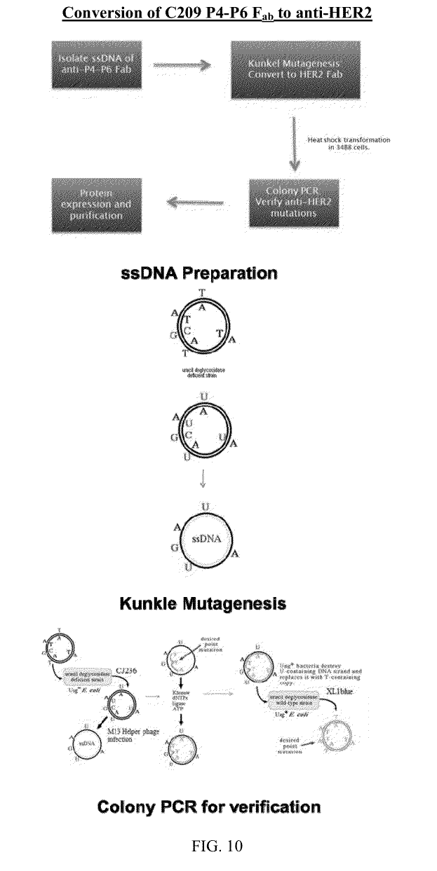

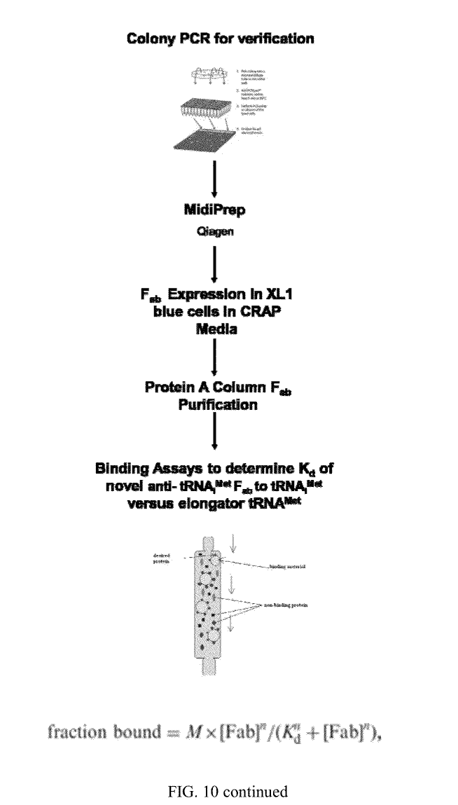

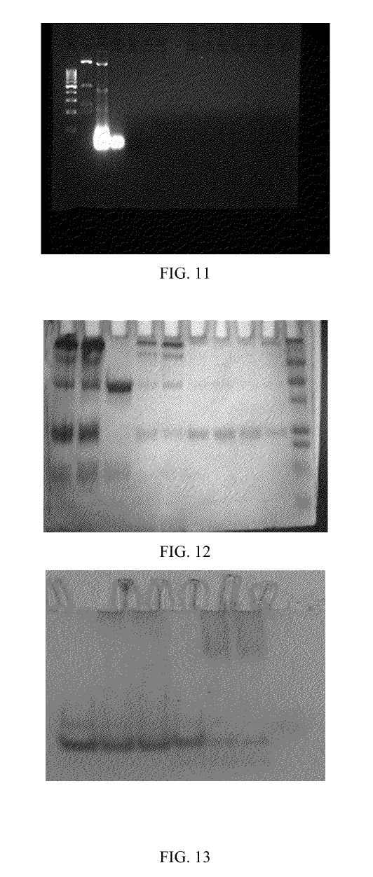

The method of selecting the anti-tRNAᵢ^{Met} Fab from a YSGR Min Library using phage display is shown in FIG. 8. The synthesis of elongator tRNA^{Met} is shown in FIG. 9.

### L. Example 3

### Targeting Oncogenic RNAs with Antibodies

Non-coding RNAs (ncRNAs) have been recognized as important cancer disease markers and therapeutic targets. The ncRNAs often form extensive structures and exert their oncogenic functions by interacting with protein partners, making the RNA-protein interfaces important targets for cancer therapy. However, currently there is a lack of a robust and general approach in targeting structured RNAs. The long-term goal is to develop antibody-based therapeutic drugs targeting structured oncogenic RNAs at the RNA-protein interfaces. Fabs can be engineered to bind RNA structure specifically and block the key RNA-protein interactions important in cancer cell proliferation and tumorigenesis. The present study develops potent Fab (antigen binding fragments) libraries for structured RNA recognition and uses phage display to select specific Fabs binding to a model oncogenic ncRNA, initiator tRNA, for potential cancer pathway mediation. Recently, overexpression of human initiator tRNA (fully conserved in all vertebrates) was shown to drive cell proliferation and cause tumor formation in mice, suggesting initiator tRNA as a potential cancer drug target. To develop effective ways to select RNA-binding antibodies, key parameters can be identified for Fab-RNA recognition and applied to design libraries tailored specifically for antiRNA Fab selection. Systematic design of the variable sites and amino acid compositions in library randomization can improve the efficacy of anti-RNA Fab libraries. Specific Fabs can be obtained and characterized for their ability to bind oncogenic initiator tRNA and mediate cancer-related biological processes. In the cell-free assay, the specific Fabs can be tested for their interference in human in vitro translation. After engineered into bispecific antibodies with anti-HER2 Fab as the delivery arm, these antibodies can be delivered to HER2 positive cancer cell lines to test their anti-cancer effect. Anti-initiator tRNA Fabs can be generated for cancer drug design and can provide a robust and general therapeutic approach to target extensively structured oncogenic RNAs, which is a challenge for antisense approaches. In addition, the bispecific antibody approach employs an innovative way to achieve high efficiency and specificity in cancer cell delivery that targets intracellular antigens.

Bispecific antibodies characterization (tRNAᵢ^{Met}- and cell-binding), stoichiometry, controls, dosage, and in vivo tests Antibody specificity: results showed that one Fab is highly specific. Fab-RNA binding can be tested with soluble Fabs: Fab-RNA binding has been tested using soluble Fabs. Fabs can be generated against structured RNAs and can be delivered into cells. tRNAᵢ^{Met} is a viable anticancer target.

Non-coding RNAs (ncRNAs) have been recognized as important cancer disease markers and therapeutic targets. Many oncogenic miRNAs (oncomirs) and long intergenic ncRNAs (lincRNAs) have been shown to promote cell proliferation, and play oncogenic roles in cancer initiation or progression. The ncRNAs often exert their oncogenic functions by interacting with protein partners, making the RNA-protein interfaces important targets for cancer therapy. Targeting the RNA-protein interface with a focus on the oncogenic RNA is more advantageous than targeting its non cancer-specific protein partners, which often have normal cellular functions that can be disrupted and lead to deleterious phenotypes. However, currently there is a lack of a robust and general approach in targeting structured RNAs. Such an approach is particularly beneficial to the development of drug-like agents that interfere with cancer-specific RNA-protein interfaces for therapeutic intervention.

Antibody-based therapeutic drugs targeting structured oncogenic RNAs at the RNA-protein interfaces can be developed. Effective Fab (antigen binding fragment) libraries for structured RNA recognition can be developed and phage displays can be used to select specific Fabs binding a model oncogenic ncRNA, initiator tRNA, for potential cancer pathway mediation. Recently, overexpression of human initiator tRNA (tRNAᵢ^{Met}, fully conserved in all vertebrates) was shown to drive cell proliferation and cause tumor formation in mice, indicating tRNAᵢ^{Met} as a cancer drug target. Fabs can be engineered to bind RNA structure specifically and block the key RNA protein interactions important in cancer cell proliferation and tumorigenesis. Specific Fabs have been selected against more than a dozen different RNAs and two crystal structures were published as Fab-RNA complexes. Unlike topologically rugged surfaces employed by other RNA-binding proteins which often evolve to only bind selected RNA structures, Fabs bind RNA with a relatively smooth, shallow, and unbiased (therefore, general) surface presented by their complementarity determining regions (CDRs, regions on Fabs binding their antigens). Additional rationale of using antibody Fabs to target oncogenic RNAs is that humanized antibodies have been widely and successfully used as therapeutic drugs with annual global sales that totaled 44 billion USD in 2010.

The ncRNAs fulfill a wide range of functions, including chromatin organization, transcriptional termination, splicing, RNA editing and processing, translational inhibition, and epigenetic regulation. The importance of ncRNAs in these key cellular processes has rendered them valuable and sometimes irreplaceable drug targets. Numerous lincRNAs have been shown to lead to tumorigenesis. HOT AIR, for instance, targets polycomb repressive complex 2 (PRC2) genome-wide to alter H3K27 methylation and gene expression patterns, resulting in increased cancer invasiveness and metastasis in breast carcinomas. Overexpression and amplification of miRNAs that target tumor-suppressor genes can also lead to cancer development. One of the well-studied oncomir clusters, miR-17-92, has been shown to be overexpressed in human lung cancers. Mediating the biogenesis of its oncomir product miR18a can be achieved by blocking the apical loop of miR18a in the primary polycistron, indicating primary miRNA as a potential cancer drug target. Recently, overexpression of tRNAᵢ^{Met} was shown to drive cell proliferation, cause malignant transformation of immortalized mouse embryonic fibroblasts (MEF) and stimulate tumor formation in mice. Most of these ncRNAs are highly structured and interact with proteins to carry out their biological functions, making RNA-protein interfaces superior targets for therapeutic intervention. However, targeting structured RNAs has been sporadic and there lacks a systematic way of producing RNA-binding agents to mediate RNA-protein interfaces. Harnessing the pro-drug property of the antibodies, RNA antibody technology is developed and its application in generation of antibodies binding specifically to oncogenic tRNAᵢ^{Met} is demonstrated. Anti-initiator tRNA Fabs can be generated for cancer drug design and the methods developed can be applied to target other structured oncogenic RNAs as well. Application of targeting structured RNAs with antibodies can be used to develop variety of biochemical tools, such as RNA crystallization chaperones', RNA immunoprecipitation, and RNA cellular imaging.

RNAs are intrinsically different from proteins in their surface functional groups and secondary and tertiary structures, current methods of targeting ncRNAs are severely limited. Although RNA interferences have been used successfully to target several long ncRNAs in the laboratory setting, targeting them in vivo is very difficult because of their extensive secondary and tertiary structures. Furthermore, off-target effects, susceptibility to cellular nuclease activity, potential immunological response, and challenges in cancer-specific delivery of the siRNAs as well as other antisense nucleotide approaches have set major hurdles in developing viable siRNA-based or general antisense-based drugs. This study uses an approach of targeting structured oncogenic RNAs with antibody fragments. The studies indicate that antibody phage display can be an effective approach for RNA targeting. As humanized antibodies have been used widely and successfully as cancer therapeutic drugs, the humanized Fab scaffold used in this library design can provide a convenient base for drug design. In addition, the bispecific antibody approach employs an innovative way to achieve high efficiency and specificity in cancer cell delivery that targets intracellular antigens.

### 1. Methods to Select for RNA-Binding Antibodies; Parameters for Fab-RNA Recognition; and Libraries Tailored for Anti-RNA Fab Selection

As there is a lack of a robust method of targeting structured ncRNAs, ncRNAs can be targeted with antibody Fabs for drug development. Phage display techniques will be used to select anti-RNA Fabs from a synthetic naive library. Synthetic Fab libraries are generally better than libraries from natural immune systems because they are unbiased toward any given antigen structure, allowing successful selection for diversified targets. In addition, synthetic Fab libraries use a defined Fab scaffold, allowing improvement of the library through CDR reengineering as well as consistent overexpression of the Fab proteins. CDR design is essential to the success of synthetic Fab libraries in phage display selections. Sophisticated design of the variable sites and amino acid composition in CDR randomization can lead to a general anti-RNA Fab library capable of producing Fab binders for most structured RNAs. Libraries can be designed to test the variable sites and amino acid composition at the Fab-RNA interfaces. The Fabs at the Fab-RNA interfaces can then be used to design next generation anti-RNA Fab libraries. With two to three rounds of optimization, a Fab library can be constructed that can be generally applicable for selection against all structured RNAs.

It has been demonstrated that Fab libraries with CDRs randomized by a preselected “reduced genetic code” instead of all 20 amino acids produce high-affinity and high-specificity Fabs against protein antigens. This design allows expansion of the randomized sites in phage display libraries without significantly increasing the theoretical library size, leading to functionally enriched libraries. The YSGX library and the YSGRX library have been applied to anti-RNA Fab selection. However, these libraries were not designed for RNA antigens, careful design needs to be employed to improve the efficacy of Fab libraries toward RNA antigens. Construction of the YSGR Min library for optimization of variable sites can be performed as discussed in Example 1.

### 2. Specific Fabs Binding to Initiator tRNA and Uses of these Fabs as Mediators of Biological Processes Related to Cancer

Overexpression of RNA polymerase III transcripts has been observed in a wide range of tumors including breast, cervix, esophagus, lung, ovary, parotid, and tongue. One type of its transcripts, tRNAs were shown to be overexpressed consistently in human ovarian cancers by RT-PCR analysis. In breast tumors versus normal breast tissues, both nuclear- and mitochondrial-encoded tRNAs were shown to be up to 10-fold higher. Furthermore, overexpression of tRNAs is not just a by-product of the carcinogenic transformation: modest overexpression of tRNAᵢ^{Met} was shown to drive cell proliferation, cause malignant transformation of immortalized MEF cells and stimulate tumor formation in mice. These results indicate that tRNAᵢ^{Met} can be a potential therapeutic target for breast cancer. tRNAᵢ^{Met} can be targeted with synthetic antibodies. Specific Fabs binding tRNAᵢ^{Met} can be selected and their roles in mediating tRNAᵢ^{Met} involved biological processes related to cancer can be demonstrated.

Using immortalized MEF cells stably transfected with tRNAᵢ^{Met} gene showed that modest overexpression of tRNAᵢ^{Met} can significantly increase protein synthesis (for c-Myc and Cyclin D1), drive cell proliferation and cause focus formation while MEF cells transfected with empty vector or elongator tRNA (tRNAₑ^{Met}) failed to do so. Furthermore, injection of tRNAᵢ^{Met} transfected MEF cells caused mice to form tumors between weeks 6-12 while the control mice with empty vector or tRNAₑ^{Met} showed no signs of tumor even after seven months. This indicates that tRNAᵢ^{Met} can be a potential candidate for a diagnostic marker and therapeutic target. The oncogenic role of tRNAᵢ^{Met} is directly correlated to protein synthesis. As protein comprises 80%-90% of a cell's dry mass, translation rate plays a dominant role in cell growth and proliferation. The rate determining step of translation is translation initiation, which depends on the availability of tRNAᵢ^{Met} and its binding to the small ribosomal subunit. The importance of translation hyperactivity in cell proliferation and transformation is supported by the oncogenicity of eEF1A, which recruits amino-acylated tRNAs to the ribosome in both translation initiation and elongation. The oncogenicity of tRNA methyltransferase Misu in breast and colon cancers and tRNA synthetase LARS1 in lung cancer indicate that aberrant tRNA maturation can induce malignant transformation as well. These observations indicate the validity of targeting tRNAᵢ^{Met} for cancer therapy as it can have multiple benefits in blocking posttranscriptional modification, amino acid charging, and ribosomal binding (FIG. 1).

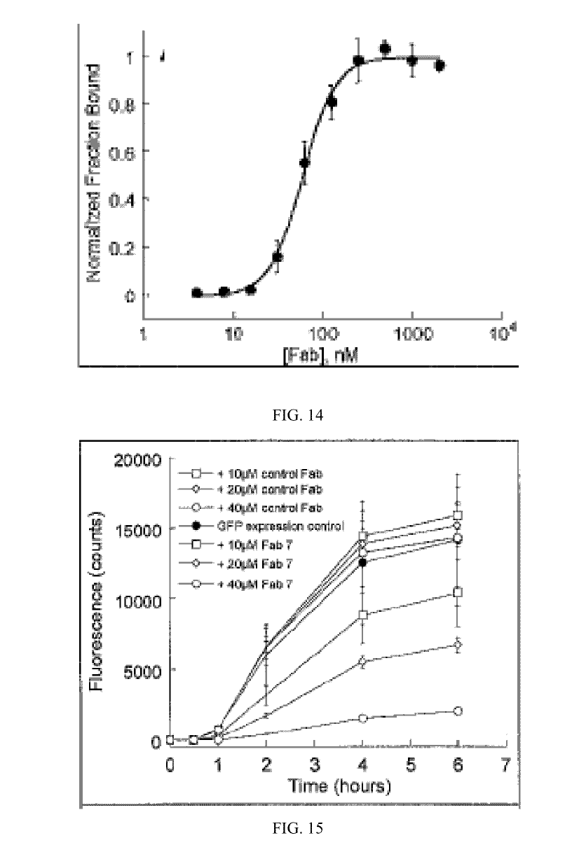

The human tRNAᵢ^{Met} gene is constructed with PCR method from DNA oligonucleotides. The DNA genes are cloned into pUC19 vector and amplified by JM109 cells. tRNAᵢ^{Met} transcript is prepared by in vitro transcription and purified by dPAGE. For selection, a 29 nt tag was engineered at the 3′ of the RNA, which allowed immobilization on the solid support after complexing with complementary biotinylated DNA oligonucleotides. Phage displayed Fab selection was carried out using published protocols. Initially Fab 1 was obtained from the YSGRX library with binding indicted by competitive phage ELISA. However, Fab 1 showed little binding to tRNAᵢ^{Met} when expressed as soluble proteins. Selection was then carried out with YSGR min library and obtained Fab7 which binds tRNAᵢ^{Met} with an affinity of 59 nM as a soluble Fab measured by filter binding assay (Table 3, FIG. 14A). Filter binding assays showed that Fab7 does not bind a control RNA, P4-P6. Native gel shift assays showed that Fab7 formed a discrete complex with tRNAᵢ^{Met} but not with E. coli tRNA mix or P4-P6. To further analyze the specificity of Fab7, methionine elongator tRNA, tRNAₑ^{Met}, and found that Fab7 does not bind tRNAₑ^{Met} in either native gel shift assay or filter binding assay. Thus, Fab7 binds tRNAᵢ^{Met} with high specificity in addition to high affinity.

TABLE 3
Affinities and amino acid sequences of specific Fabs binding
initiator tRNA

| | | K_{d} | | | | | | |
|---|---|---|---|---|---|---|---|---|
| Target | clone | (nM) | L1 | L2 | L3 | H1 | H2 | H3 |
| tRNAᵢ^{Met} | 7 | 59 ± 3 | SSRYR | YGAYRLSSGVPYR | GSSYPV | NFSGSGI | GSGSSRGYTR | SGSGSRYAL |

Using 1-step human in vitro protein expression kit (Thermo Scientific), the effect of Fab7 on GFP reporter expression efficiency was investigated. Results (FIG. 15) showed that Fab7 significantly reduced the GFP production in a concentration dependent manner, while a control Fab had no effect, indicating the ability of Fab7 to inhibit translation in vitro.

i. Selection, Expression, and Characterization of tRNA,Met_Binding Fabs in Cell-Free Assays.

The selection of anti-tRNAᵢ^{Met} Fabs can be performed using other libraries or repeat with existing libraries to obtain more Fabs. Fabs with varied affinities can be generated from affinity maturation mutation of Fab7. These Fabs can be expressed, and their affinities and specificities can be analyzed with filter binding and native gel shift assays. A solution based structure probing approach, the hydroxyl radical footprinting assay, can be used to reveal the binding site of Fab on the tRNA, which helps predict the possible tRNA metabolic pathways that anti-tRNA Fabs can interfere with. Specific Fabs can also be tested for their ability to reduce the efficiency of in vitro translation as carried out with Fab7. The effect of the affinities on in vitro translation efficiency can be obtained, which provide a basis for live cell assays.

ii. Investigating the Function of Anti-tRNA Fabs in Cell-Based Assays.

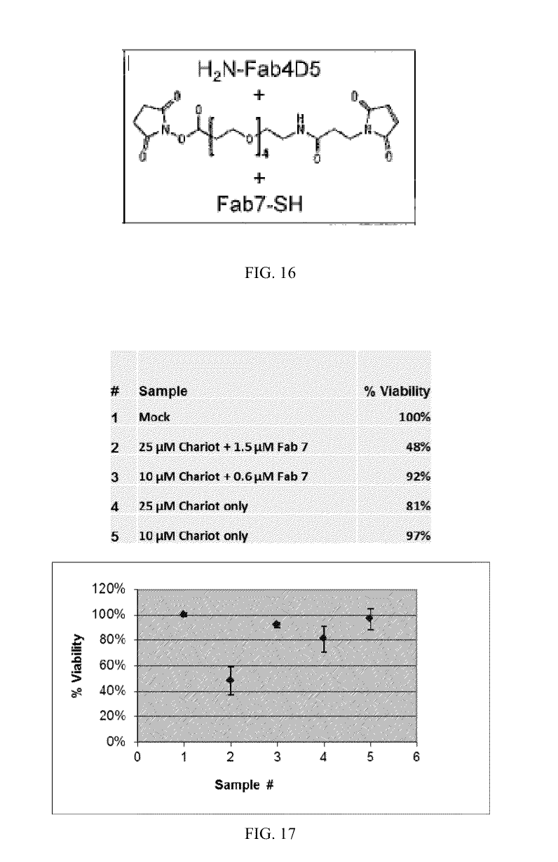

Therapeutic antibodies have been delivered into cancer cells with various efficiencies and specificities via conjugation or complexation approaches. To further aid in the delivery efficiency and specificity of antibody fragments into cancer cells and tumors, the anti-tRNAᵢ^{Met} Fabs can be engineered into bispecific antibodies. Holding great promise, bispecific antibodies serve two critical functions: one Fab (therapeutic arm) to recognize the oncogenic RNA to achieve therapeutic function and the other Fab (delivery arm) to achieve specific and efficient cancer cell delivery via cancer cell markers and endocytosis. Multiple forms of bispecific antibodies have been created by research labs and pharmaceutical companiesBispecific Fab′2 antibodies (FIG. 2) can be generated via an amine-to-sulfhydryl crosslinker (FIG. 16). Readily available through CDR swapping on the Fab scaffold, anti-HER2 Fab4D5 can be used as the delivery arm since the HER2-binding antibody has been used successfully in delivering antisense drugs into breast tumor in a mouse model. Fab4D5 can be first coupled with the crosslinker via amino-NHS ester chemistry and then reacted with free sulfhydryl-containing Fab7 via maleimide moiety. As all the naturally occurring cysteines of Fab7 are in the disulphide bond form, adding a free Cys to the C-terminus can result in a lot of Fab dimer formation. A heavy chain A121C mutant of Fab7 expressed relatively well without significant amount of dimer formation. Results showed reasonably good yield of the bispecific Fabs through the chemistry described in FIG. 16. Under current conditions, a mixture of coupled antibodies were obtained, ˜60% 1:1 and ˜40% 1:2 (Fab4D5:Fab7) conjugates. While both forms can have in vivo effect, gel filtration can be used to separate the mixture to allow accurate evaluation of each form. Native gel can also be used to verify the binding between these bispecific Fabs and tRNAᵢ^{Met} before cell-based assays.

Bispecific Fabs can be prepared using three versions of Fab4D5, version 2, 4, and 8, corresponding to HER2-binding affinities of 4.7, 0.82, and 0.1 nM measured in the IgG format. The different affinities can affect the cell binding, internalization and endosome escape, which will be evaluated through confocal microscopy using FITC-labeled bispecific Fabs. Internalization into HER2 positive but not into HER2 negative cell lines can be verified. Specifically SK-BR-3 and MDA-MB-231 breast cancers can be used as HER2 positive and negative cell lines, respectively.

Cell based assays can be used to determine the efficacy of bispecific Fabs (initial dosage: 20 nM-10 μM) in their ability to distinguish between HER2+ and HER2− cell lines, and compared to their monospecific Fab controls. The bispecific Fabs can show inhibition on cell growth, and this effect can be limited to only HER2 positive cells. CellTiter 96 A_{Queous} assay (MTS, Promega) can be the method to identify the effect of the bispecific Fabs on cell viability. Metabolic labeling using ³⁵S-methionine can be used to directly measure changes in protein expression with and without treatment of bispecific Fabs and their respective controls using standard protocols. Similar assays can be performed on a pancreatic cancer cell line L3 6pl (HER2+)50 and control cell line hTERT-HPNE (HER2−) upon verification of the cell internalization similar to that described above.

Specific Fab7 binding to tRNAᵢ^{Met} have been obtained which showed concentration-dependent inhibition in translation in vitro. Fab7 and other Fabs can be engineered into bispecific antibodies with an anti-HER2 Fab arm and delivered into HER2+ cancer cell lines. If their anti-cancer activities are tested positive, they can be anti-cancer drugs.

The disclosed compositions and methods can be used to obtain a Fab library effective for essentially all structured RNA targets. Anti-cancer bispecific antibodies can also be obtained with both anti-tRNAᵢ^{Met} and anti-HER2 Fabs. Bispecific antibodies can be delivered into mouse xenograft models, characterizing their anti-cancer effect in vivo, and developing them into effective antibody cancer drugs.

### M. Example 4

### 1. Surface Entropy Reduction

Protein-Protein interaction disfavors Lysine and glutamate residues, mutating large flexible side chains with smaller amino acid residues like Alanine and Serine provides surface entropy shield preventing non-specific aggregation and precipitation. Residues to be chosen should not interfere with protein function.

Crystallization of Osp A protein—mutations based on 1OSP and 1FJ1 crystal structures. 13 mutations replacing Lysine and Glutamate: K48A, E196A, K60A, K83A, E37S, E45S, K46S,K64S,E104S,K107S,K239S,E240S and K254S (trial and error basis) can be made. Mutant Osp Asm 1 was crystallizable in a wide variety of solution conditions giving 1.15 Å resolution.

The crystal structure of Fab2-P4P6 (2R8S) was studied and residues which could reduce the surface entropy were found and they were categorized into Category A and Category B with respect to more probable candidates for mutation and less probable ones. A tool has been developed for designing surface mutations for crystallization (nihserver.mbi.ucla.edu/SER/)

The suggestions from the web-based surface entropy software are as follows.

| Cluster1 | Cluster2 | Cluster3 |
|---|---|---|
| Residues 187-190(L) | Residues 37-39(L) | Residues 217-220(H) |
| KADYEKHKVYACE | QQKPGKAPK | KKVEPK |
| SERp Score: 5.54 | SERp Score: 4.21 | SERp Score: 3.64 |
| E 187 (L) | Q 37(L) | K 217(H) |
| K 188 (L) | Q 38 (L) | K 218 (H) |
| K 190 (L) | K 39 (L) | E 220 (H) |

In one example, the final residues that were mutated were K217(H), K218(H), E220(H), K190(L), and E123(L). Kunkel mutagenesis can be used to incorporate the mutations. Since the five mutations are at different locations, three kunkel primers were used.

Mutations on Fab2 can be added using the Kunkel method. The following parameters can be used. Lys217,Lys218,Glu220 and Stop codon (heavy chain) AGCAACACCAAGGTCGACGCCGCCGTTGCCCCCAAATCTTGTGACAAACTACACAT AGGGCCGGCCCTCTGGTTCC (SEQ ID NO:37); Melting temp 5′ end=56° C.; Melting temp 3′ end=64° C. Glu123(Light chain) CTTCATCTTCCCGCCATCTGATGCCCAGTTGAAATCTGGAACTGC (SEQ ID NO:38); Melting temp 5′ end=66° C.; Melting temp 3′ end=58° C. Lys190(Light chain) AGCAGACTACGAGAAACACGCCGTCTACGCCTGCGAAGTA (SEQ ID NO:39); Melting temp 5′ end=56° C.; Melting temp 3′ end=60° C. GCC is a frequent Alanine coding triplet, AGC is a frequent Serine coding triplet.

The Kunkel method further comprises performing a Mini-prep to extract mutated DNA. These were sent for sequencing, checked for incorporation of mutations and pooled together. Kunkel Mutagenesis was also performed for second set of Serine mutations yielding P4P6Fab2SMS. The first Kunkel for Serine mutations had mis-incorporations in light chain. A single strand extraction of mutated heavy chain was performed and then mutations for light chain were performed. Mini-prep DNA were sent for sequencing, checked for incorporation of mutations and pooled together. Kunkel mutagenesis was performed on P4P6Fab2 to incorporate the stop codon. Positive clones were then identified by colony PCR, DNA was extracted by mini-prep and pooled together.

### 2. Optimization of CRAP Media

Optimizing the yield of all the three Fabs (around 1.5 mg/L of CRAP media) can be performed. Various trials were conducted to increase the yield of the Fabs. Previous protocols used 25 ml 2YT/Ampicillin starting culture at 37° C. O/N; 24 hrs incubation IL CRAP/Ampicillin culture in 2.8 L baffled flask at 30° C. 250 RPM.

The optimized protocol and contain 25 ml 2YT/Ampicillin starting culture at 30° C. O/N; and 24 hrs incubation 500 ml CRAP/Ampicillin culture in 2.8 L baffled flask at 30° C. 300 RPM. The indications for a good yield are after 24 hrs OD600 nm >6 and good observable foaming.

| P4PFab2SMA | 30° C. Starting culture | 37° C. starting culture |
|---|---|---|
| O/N OD_{600 nm} | 7.117 | 8.6276 |
| After 24 hr OD_{600 nm} | 10.485 | 5.989 |
| Protein expression | 2.12 mg/L | 1.14 mg/L |

CRAP media can contain the following components:

| Media components | 500 ml | |
|---|---|---|
| (NH₄)₂SO₄ | 1.785 | g |
| NaCitrate-2H₂0 | 0.355 | g |
| KCl | 0.535 | g |
| Yeast extract | 2.68 | g |
| HyCase SF Casein | 2.68 | g |
| dd H₂0 | 436 | ml |

Adjust pH to 7.3, autoclave and then add the following (these solutions should be filtered sterile) to cooled CRAP media (room temperature).

| Media components | 500 ml |
|---|---|
| 1M MOPS, pH 7.3 | 55 ml |
| 50% glucose | 5.5 ml |
| 1M MgSO₄ | 3.5 ml |
| 1000x Ampicillin (100 mg/ml) | 0.5 ml |

### 3. Crystallization

Crystal screening can be performed using Hampton crystal screening kit (96) and Index kit (96) for P4P6-P4P6Fab2, P4P6-P4P6Fab2SMA and PfP6-P4P6Fab2SMS complexes. The crystal screening conditions can be sample concentration: 12 mg/ml; sample buffer: 10 mM Tris pH7.5, 25 mM Mgcl2 50 mM Nacl, 0.5 mM; Spermine-4HCl and RNase inhibitor, Reservoir volume: 1001 Temperature: 4° C. and 22° C.; Drop volume −1 μl Sample: 0.5 μl and Reservoir: 0.5 μl

A list of crystal hits for the ΔC209P4P6 RNA binding to Fab2 and its mutants is provided below.

| RNA-Fab Complex | Solution buffer condition | Reservoir condition | Temp | Crystal form |
|---|---|---|---|---|
| ΔC209P4P6Fab2SMS | 10 Mm Tris | 2.0M NaCl, | 20° C. | Single |
| | 7.5, 25 Mm | 105 w/v | | crystals |
| | MgCl₂, 50 Mm | PEG 6000 | | |
| | NaCl, 0.5 Mm | | | |
| | Spermine•4HCl | | | |
| ΔC209P4P6Fab2SMA | 10 Mm Tris | 0.15M DL-Malic | 20° C. | Rosettes |
| | 7.5, 25 Mm | acid pH 7.0, 20% | | and |
| | MgCl₂, 50 Mm | w/v | | plates |
| | NaCl, 0.5 | PEG 3,350 | | |
| | Spermine•4HCl | | | |
| ΔC209P4P6Fab2SMA | 10 Mm Tris | 0.2M Magnesium | 20° C. | Rosettes |
| | 7.5, 25 Mm | chloride | | and |
| | MgCl₂, 50 Mm | hexahydrate, | | plates |
| | NaCl, 0.5 Mm | 0.1M Tris Ph | | |
| | Spermine•4HCl | 8.5, 3.4M | | |
| | | 1,6-Hexanediol | | |
| ΔC209P4P6Fab2SMS | 10 Mm Tris | 2.0M NaCl, | 20° C. | Single |
| | 7.5, 25 Mm | 105 w/v | | crystals |
| | MgCl₂, 50 Mm | PEG 6000 | | |
| | NaCl, 0.5 Mm | | | |
| | Spermine•4HCl | | | |
| ΔC209P4P6Fab2SMA | 10 Mm Tris | 0.15M DL-Malic | 20° C. | Rosettes |
| | 7.5, 25 Mm | acid pH 7.0, | | and |
| | MgCl₂, 50 Mm | 20% w/v | | plates |
| | NaCl, 0.5 Mm | PEG 3,350 | | |
| | Spermine•4HCl | | | |
| ΔC209P4P6Fab2SMA | 10 Mm Tris | 0.01M Magnesium | 20° C. | Needles |
| | 7.5, 25 Mm | chloride hexa- | | |
| | MgCl₂, 50 Mm | hydrate, 0.05M | | |
| | NaCl, 0.5 Mm | MES monohydrate | | |
| | Spermine•4HCl | ph 5.6, 1.8M | | |
| | | Lithium sulfate | | |
| | | monohydrate | | |
| ΔC209P4P6Fab2SMS | 10 Mm Tris | 0.2M Ammonium | 4° C. | Plates |
| | 7.5, 25 Mm | acetate, 0.1M | | |
| | MgCl₂, 50 Mm | Sodium citrate | | |
| | NaCl, 0.5 Mm | tribasic dihy- | | |
| | Spermine•4HCl | drate pH 5.6, | | |
| | | 30% v/v | | |
| | | (+/−)-2-Methyl-2,4- | | |
| | | pentanediol | | |
| ΔC209P4P6Fab2SMA | 10 Mm Tris | 0.2M Sodium | 4° C. | Plates |
| | 7.5, 25 Mm | citrate tribasic | | |
| | MgCl₂, 50 Mm | dihydrate, 0.1M | | |
| | NaCl, 0.5 Mm | HEPES sodium pH | | |
| | Spermine•4HCl | 7.5, 30% v/v | | |
| | | (+/−)-2-Methyl-2,4- | | |
| | | pentanediol | | |
| ΔC209P4P6Fab2SMS | 10 Mm Tris | 0.2M Sodium | 4° C. | Plates |
| | 7.5, 25 Mm | citrate tribasic | | |
| | MgCl₂, 50 Mm | dihydrate, 0.1M | | |
| | NaCl, 0.5 Mm | HEPES sodium pH | | |
| | Spermine•4HCl | 7.5, 30% v/v | | |
| | | (+/−)-2-Methyl- 2,4- | | |
| | | pentanediol | | |
| ΔC209P4P6Fab2 | 10 Mm Tris | 0.2M Sodium | 4° C. | Rosettes |
| | 7.5, 25 Mm | citrate tribasic | | |
| | MgCl₂, 50 Mm | dihydrate, 0.1M | | |
| | NaCl,0.5 Mm | HEPES sodium pH | | |
| | Spermine•4HCl | 7.5, 30% v/v | | |
| | | (+/−)-2-Methyl-2,4- | | |
| | | pentanediol | | |
| ΔC209P4P6Fab2 | 10 Mm Tris | 0.2M Ammonium | 4° C. | Plates |
| | 7.5, 25 Mm | acetate, 0.1M | | |
| | MgCl₂, 50 Mm | Sodium citrate tri- | | |
| | NaCl, 0.5 Mm | basic dihydrate | | |
| | Spermine•4HCl | pH 5.6, 30% v/v | | |
| | | (+/−)-2-Methyl-2,4- | | |
| | | pentanediol | | |

Optimization results using hanging drop technique are as follows.

| | Solution buffer | Reservoir | | Crystal |
|---|---|---|---|---|
| RNA-Fab Complex | condition | condition | Temp | form |
| ΔC209P4P6Fab2SMS | 10 Mm Tris | 2.0M NaCl, | 20° C. | Single |
| | 7.5, 25 Mm | 105 w/v | | crystals |
| | MgCl₂, 50 Mm | PEG 6000 | | |
| | NaCl, 0.5 Mm | | | |
| | Spermine•4HCl | | | |
| ΔC209P4P6Fab2SMA | 10 Mm Tris | 0.15M DL-Malic | 20° C. | Rosettes |
| | 7.5, 25 Mm | acid pH 7.0, | | and |
| | MgCl₂, 50 Mm | 20% w/v | | plates |
| | NaCl, 0.5 Mm | PEG 3,350 | | |
| | Spermine•4HCl | | | |

### 4. Conclusion

Using visual inspection and web-based software 5 sites for surface entropy reduction on Fab surface were selected. Mutations were incorporated and clones were isolated. Optimisation of the yield of P4P6Fabs to 3-4 mg/L has been performed. Large scale expression of the supermutant clones P4P6Fab2SMA,P4P6Fab2SMS, parent clone P4P6Fab2 and our RNA construct P4P6 have been performed.

Initial screening of all Fabs complexed with P4P6 side by side to determine the crystal hit ratio using commercially available crystallization screening kit Hampton and Index screening and Natrix screening condition s were set up and good hit ratio has been obtained. Optimisation trays for the obtained crystal hits were set up. Crystals were fished out and sent to obtain diffraction pattern and they are found to be diffracting close to 6 A. A considerable increase in the hit ratio in these crystal screens has led to extension of this surface entropy reduction technique in Chaperone Assisted RNA Crystallography to crystallization of Fab′ s binding to FN & VC glycine riboswitches.

### REFERENCES

1. Zhang, B., Pan, X., Cobb, G. P. & Anderson, T. A. microRNAs as oncogenes and tumor suppressors. Dev Biol 302, 1-12 (2007).
2. Wang, D. et al. Human microRNA oncogenes and tumor suppressors show significantly different biological patterns: from functions to targets. PLoS One 5 (2010).
3. Tsai, M. C., Spitale, R. C. & Chang, H. Y. Long intergenic noncoding RNAs: new links in cancer progression. Cancer Res 71, 3-7 (2011).
4. Huarte, M. & Rinn, J. L. Large non-coding RNAs: missing links in cancer? Hum Mol Genet. 19, R152-61 (2010).
5. Marshall, L., Kenneth, N. S. & White, R. J. Elevated tRNA(iMet) synthesis can drive cell proliferation and oncogenic transformation. Cell 133, 78-89 (2008).
6. Ye, J. D. et al. Synthetic antibodies for specific recognition and crystallization of structured RNA. Proc Natl Acad Sci USA 105, 82-7 (2008).
7. Koldobskaya, Y. et al. A portable RNA sequence whose recognition by a synthetic antibody facilitates structural determination. Nat Struct Mol Biol 18, 100-6 (2011).
8. Ravindran, P. P., Heroux, A. & Ye, J. D. Improvement of the crystallizability and expression of an RNA crystallization chaperone. J Biochem 150, 535-43 (2011).
9. Rould, M. A., Perona, J. J., Soll, D. & Steitz, T. A. Structure of E. coli glutaminyl-tRNA synthetase complexed with tRNA(Gln) and ATP at 2.8 A resolution. Science 246, 1135-42 (1989).
10. Lu, M. & Steitz, T. A. Structure of Escherichia coli ribosomal protein L25 complexed with a 5S rRNA fragment at 1.8-A resolution. Proc Natl Acad Sci USA 97, 2023-8 (2000).
11. Kirkwood, J. M. et al. Immunotherapy of cancer in 2012. CA Cancer J Clin (2012).
12. Hayashita, Y. et al. A polycistronic microRNA cluster, miR-17-92, is overexpressed in human lung cancers and enhances cell proliferation. Cancer Res 65, 9628-32 (2005).
13. Gutschner, T., Bass, M. & Diederichs, S, Noncoding RNA gene silencing through genomic integration of RNA destabilizing elements using zinc finger nucleases. Genome Res 21, 1944-54 (2011).
14. Fellouse, F. A., Wiesmann, C. & Sidhu, S. S. Synthetic antibodies from a four-amino-acid code: a dominant role for tyrosine in antigen recognition. Proc Natl Acad Sci USA 101, 12467-72 (2004).
15. Fellouse, F. A. et al. Molecular recognition by a binary code. J Mol Biol 348, 1153-62 (2005).
16. Sidhu, S. S. et al. Phage-displayed antibody libraries of synthetic heavy chain complementarity determining regions. J Mol Biol 338, 299-310 (2004).
17. Sidhu, S. S., Lowman, H. B., Cunningham, B. C. & Wells, J. A. Phage display for selection of novel binding peptides. Methods Enzymol 328, 333-63 (2000).
18. Agris, P. F. et al. Experimental models of protein-RNA interaction: isolation and analyses of tRNA(Phe) and U snRNA-binding peptides from bacteriophage display libraries. J Protein Chem 18, 425-35 (1999).
19. Mian, I. S., Bradwell, A. R. & Olson, A. J. Structure, function and properties of antibody binding sites. J Mol Biol 217, 133-51 (1991).
20. Braunagel, M. & Little, M. Construction of a semisynthetic antibody library using trinucleotide oligos. Nucleic Acids Res 25, 4690-1 (1997).
21. Cabarcas, S. & Schramm, L. RNA polymerase III transcription in cancer: the BRF2 connection. Mol Cancer 10, 47 (2011).
22. Winter, A. G. et al. RNA polymerase III transcription factor TFIIIC2 is overexpressed in ovarian tumors. Proc Natl Acad Sci USA 97, 12619-24 (2000).
23. Pavon-Eternod, M. et al. tRNA over-expression in breast cancer and functional consequences. Nucleic Acids Res 37, 7268-80 (2009).
24. Zetterberg, A. & Killander, D. Quantitative cytophotometric and autoradiographic studies on the rate of protein synthesis during interphase in mouse fibroblasts in vitro. Exp Cell Res 40, 1-11 (1965).
25. Francis, M. A. & Rajbhandary, U. L. Expression and function of a human initiator tRNA gene in the yeast Saccharomyces cerevisiae. Mol Cell Biol 10, 4486-94 (1990).
26. Tomlinson, V. A. et al. Translation elongation factor eEF 1A2 is a potential oncoprotein that is overexpressed in two-thirds of breast tumours. BMC Cancer 5, 113 (2005).
27. Frye, M. & Watt, F. M. The RNA methyltransferase Misu (NSun2) mediates Myc-induced proliferation and is upregulated in tumors. Curr Biol 16, 971-81 (2006).
28. Shin, S. H. et al. Implication of leucyl-tRNA synthetase 1 (LARS1) over-expression in growth and migration of lung cancer cells detected by siRNA targeted knock-down analysis. Exp Mol Med 40, 229-36 (2008).
29. Wong, I. & Lohman, T. M. A double-filter method for nitrocellulose-filter binding: application to protein-nucleic acid interactions. Proc Natl Acad Sci USA 90, 5428-32 (1993).
30. Cochrane, J. C., Batey, R. T. & Strobel, S. A. Quantitation of free energy profiles in RNA-ligand interactions by nucleotide analog interference mapping. Rna 9, 1282-9 (2003).
31. Sherman, E. M., Esquiaqui, J., Elsayed, G. & Ye, J. D. An energetically beneficial leader-linker interaction abolishes ligand-binding cooperativity in glycine riboswitches. Rna 18, 496-507 (2012).
32. Guillon, L., Schmitt, E., Blanquet, S. & Mechulam, Y. Initiator tRNA binding by e/aIF5B, the eukaryotic/archaeal homologue of bacterial initiation factor IF2. Biochemistry 44, 15594-601 (2005).
33. Hamrita, B. et al. An elongation factor-like protein (EF-Tu) elicits a humoral response in infiltrating ductal breast carcinomas: An immunoproteomics investigation. Clin Biochem 44, 1097-104 (2011).
34. Cassinelli, G. et al. Inhibition of c-Met and prevention of spontaneous metastatic spreading by the 2-indolinone RPI-1. Mol Cancer Ther 5, 2388-97 (2006).
35. Morris, M. C., Depollier, J., Mery, J., Heitz, F. & Divita, G. A peptide carrier for the delivery of biologically active proteins into mammalian cells. Nat Biotechnol 19, 1173-6 (2001).
36. Schwarze, S. R. & Dowdy, S. F. In vivo protein transduction: intracellular delivery of biologically active proteins, compounds and DNA. Trends Pharmacol Sci 21, 45-8 (2000).
37. Rizk, S. S. et al. An engineered substance P variant for receptor-mediated delivery of synthetic antibodies into tumor cells. Proc Natl Acad Sci USA 106, 11011-5 (2009).
38. Bale, S. S. et al. Nanoparticle-mediated cytoplasmic delivery of proteins to target cellular machinery. ACS Nano 4, 1493-500 (2010).
39. Iyer, A. K. et al. Novel human single chain antibody fragments that are rapidly internalizing effectively target epithelioid and sarcomatoid mesotheliomas. Cancer Res 71, 2428-32 (2011).
40. Holmes, D. Buy buy bispecific antibodies. Nat Rev Drug Discov 10, 798-800 (2011).
41. Thakur, A. & Lum, L. G. Cancer therapy with bispecific antibodies: Clinical experience. Curr Opin Mol Ther 12, 340-9 (2010).
42. Chames, P. & Baty, D. Bispecific antibodies for cancer therapy: the light at the end of the tunnel? MAbs 1, 539-47 (2009).
43. Loo, L., Robinson, M. K. & Adams, G. P. Antibody engineering principles and applications. Cancer J 14, 149-53 (2008).
44. Jung, G., Honsik, C. J., Reisfeld, R. A. & Muller-Eberhard, H. J. Activation of human peripheral blood mononuclear cells by anti-T3: killing of tumor target cells coated with anti-target-anti-T3 conjugates. Proc Natl Acad Sci USA 83, 4479-83 (1986).
45. Eigenbrot, C., Randal, M., Presta, L., Carter, P. & Kossiakoff, A. A. X-ray structures of the antigen-binding domains from three variants of humanized anti-p185HER2 antibody 4D5 and comparison with molecular modeling. J Mol Biol 229, 969-95 (1993).
46. Wang, Y. et al. Tumor delivery of antisense oligomer using trastuzumab within a streptavidin nanoparticle. Eur J Nucl Med Mol Imaging 36, 1977-86 (2009).
47. Moor, N. A., Ankilova, V. N. & Lavrik, O. I. Recognition of tRNAPhe by phenylalanyl-tRNA synthetase of Thermus thermophilus. Eur J Biochem 234, 897-902 (1995).
48. Peterson, E. T. & Uhlenbeck, O. C. Determination of recognition nucleotides for Escherichia coli phenylalanyl-tRNA synthetase. Biochemistry 31, 10380-9 (1992).
49. Harrington, K. M., Nazarenko, I. A., Dix, D. B., Thompson, R. C. & Uhlenbeck, O. C. In vitro analysis of translational rate and accuracy with an unmodified tRNA. Biochemistry 32, 7617-22 (1993).
50. Curnow, A. W., Kung, F. L., Koch, K. A. & Garcia, G. A. tRNA-guanine transglycosylase from Escherichia coli: gross tRNA structural requirements for recognition. Biochemistry 32, 5239-46 (1993).
51. Rudnick, S. I. et al. Influence of affinity and antigen internalization on the uptake and penetration of Anti-HER2 antibodies in solid tumors. Cancer Res 71, 2250-9 (2011).
52. Rudnick, S. I. & Adams, G. P. Affinity and avidity in antibody-based tumor targeting.

1.-51. (canceled)

52. An antibody or fragment thereof, comprising: at least one Fab that specifically binds to tRNAᵢ^{Met},
wherein the isolated antibody or fragment thereof comprises a heavy chain and a light chain,
wherein variable regions of the heavy chain and the light chain comprise complementarity determining regions (CDRs), and
wherein the CDRs of the light chain comprises one or more of amino acid sequences SSRYR (SEQ ID NO:1), YGAYRLSSGVPYR (SEQ ID NO:2), and GSSYPV (SEQ ID NO:3).

53. The antibody or fragment thereof of claim 52, wherein the CDRs of the heavy chain comprise one or more of amino acid sequences NFSGSGI (SEQ ID NO:4), GSGSSRGYTR (SEQ ID NO:5), and SGSGSRYAL (SEQ ID NO:6).

54. The antibody or fragment thereof of claim 52, wherein the CDRs of the light chain comprise amino acid sequence SSRYR (SEQ ID NO:1), YGAYRLSSGVPYR (SEQ ID NO:2), and GSSYPV (SEQ ID NO:3), and wherein the CDRs of the heavy chain comprise amino acid sequences NFSGSGI (SEQ ID NO:4), GSGSSRGYTR (SEQ ID NO:5), and SGSGSRYAL (SEQ ID NO:6).

55. The antibody or fragment thereof of claim 52, wherein the antibody or fragment thereof is monoclonal.

56. The antibody or fragment thereof of claim 52, wherein the antibody or fragment thereof is humanized.

57. The antibody or fragment thereof of claim 52, wherein the antibody or fragment thereof inhibits cell proliferation, blocks binding of tRNAᵢ^{Met} with elongation factors, or prevents tRNAᵢ^{Met} from initiating polypeptide synthesis.

58. A bacteriophage, comprising: a nucleic acid encoding the antibody or fragment thereof of claim 52.

59. A bacteriophage, comprising: a nucleic acid encoding the antibody or fragment thereof of claim 53.

60. A bacteriophage, comprising: a nucleic acid encoding the antibody or fragment thereof of claim 54.

61. A bi-specific Fab complex comprising a therapeutic arm and a delivery arm, wherein the therapeutic arm comprises an anti-tRNAᵢ^{Met} Fab.

62. The bi-specific Fab complex of claim 61, wherein the anti-tRNAᵢ^{Met} Fab comprises a heavy chain and a light chain,
wherein variable regions of the heavy chain and the light chain comprise complementarity determining regions (CDRs), and
wherein the CDRs of the light chain comprise one or more of amino acid sequences SSRYR (SEQ ID NO:1), YGAYRLSSGVPYR (SEQ ID NO:2), and GSSYPV (SEQ ID NO:3).

63. The bi-specific Fab complex of claim 62, wherein the CDRs of the heavy chain comprise one or more of amino acid sequences NFSGSGI (SEQ ID NO:4), GSGSSRGYTR (SEQ ID NO:5), and SGSGSRYAL (SEQ ID NO:6).

64. The bi-specific Fab complex of claim 62, wherein the CDRs of the light chain comprise amino acid sequences SSRYR (SEQ ID NO:1), YGAYRLSSGVPYR (SEQ ID NO:2), and GSSYPV (SEQ ID NO:3), and wherein the CDRs of the heavy chain comprise amino acid sequences NFSGSGI (SEQ ID NO:4), GSGSSRGYTR (SEQ ID NO:5), and SGSGSRYAL (SEQ ID NO:6).

65. The bi-specific Fab complex of claim 61, wherein the delivery arm comprises an anti-HER2 Fab.

66. The bi-specific Fab complex of claim 61, wherein the anti-tRNAᵢ^{Met} Fab inhibits cell proliferation, blocks binding of tRNAᵢ^{Met} with elongation factors, or prevents tRNAᵢ^{Met} from initiating polypeptide synthesis.

67. A phage display library, comprising: bacteriophage comprising a nucleic acid that encodes an antibody or fragment thereof,
wherein the antibody or fragment thereof comprises a heavy chain and a light chain,
wherein variable regions of the heavy chain and the light chain comprise mutated complementarity determining regions (CDRs), and
wherein the mutated CDRs comprise selectively randomized Tyrosines, Serines, Glycines, and Arginines.

68. The phage display library of claim 67, wherein the mutated CDRs do not contain consecutive Arginines.

69. The phage display library of claim 67, wherein the CDRs of the light chain comprise one or more of amino acid sequences SSRYR (SEQ ID NO:1), YGAYRLSSGVPYR (SEQ ID NO:2), and GSSYPV (SEQ ID NO:3), and wherein the CDRs of the heavy chain comprise one or more of amino acid sequences NFSGSGI (SEQ ID NO:4), GSGSSRGYTR (SEQ ID NO:5), and SGSGSRYAL (SEQ ID NO:6).

70. The phage display library of claim 67, wherein the CDRs of the light chain comprise amino acid sequences SSRYR (SEQ ID NO:1), YGAYRLSSGVPYR (SEQ ID NO:2), and GSSYPV (SEQ ID NO:3), and wherein the CDRs of the heavy chain comprise amino acid sequences NFSGSGI (SEQ ID NO:4), GSGSSRGYTR (SEQ ID NO:5), and SGSGSRYAL (SEQ ID NO:6).

71. The phage display library of claim 67, wherein the nucleic acid encoding the mutated CDRs comprises two degenerate codons applied in an alternating fashion,
wherein the first degenerate codon is TMT, wherein M is A or C, and wherein equal portions of Tyrosine and Serine are encoded, and
wherein the second degenerate codon is VGT, wherein V is A, C, or G, and wherein equal portions of Serine, Glycine, and Arginine are encoded.
